(19) United States
(12) Patent Application Publication
(10) Pub. No.: US 2012/0149732 A1
(43) Pub. Date: Jun. 14, 2012

(54) MULTIFUNCTIONAL LINKERS AND METHODS FOR THE USE THEREOF

(76) Inventors: Alexander Chucholowski, San Diego, CA (US); Alisher Khasanov, Escondido, CA (US); Gregory Parker, Cardiff by the Sea, CA (US); Tong Zhu, San Diego, CA (US)

(21) Appl. No.: 13/215,109

(22) Filed: Aug. 22, 2011

Related U.S. Application Data
(63) Continuation-in-part of application No. 12/968,225, filed on Dec. 14, 2010.

(51) Int. Cl. A61K 31/4545; C07F 7/18; C07D 213/71; C07D 401/12; C07D 305/14; A61K 31/4402; A61K 31/337; A61K 31/4025; A61P 35/00; A61P 29/00; A61P 31/00; A61P 37/00; A61P 37/08; A61P 25/00; A61P 17/00; A61P 9/00; A61P 3/10; A61P 1/00; C07F 7/12

(52) U.S. Cl. 514/318; 556/414; 546/14; 552/505; 540/4; 546/291; 546/193; 549/511; 548/525; 514/346; 514/449; 514/422; 549/510

(57) ABSTRACT

In accordance with the present invention, novel multifunctional compounds have been developed which have orthogonal reactive groups thereon, thereby facilitating preparation of compounds having multiple functional properties (e.g., a targeting moiety and a biologically active moiety). Such constructs are useful for a variety of applications, e.g., for the delivery of biologically compatible materials, and release thereof in active form. Therefore, in accordance with the present invention, there are provided multifunctional linkers of defined structure, as well as various derivatives thereof bearing one or more biologically active components thereon. Also provided in accordance with the present invention are methods for the preparation of such constructs, as well as various uses thereof.

### RELATED APPLICATIONS

This application is a continuation-in-part of U.S. Ser. No. 12/968,225, filed Dec. 14, 2010, now pending, the entire contents of which are hereby incorporated by reference herein.

### FIELD OF THE INVENTION

The present invention relates to multifunctional linkers having orthogonal reactive groups thereon, thereby facilitating delivery of biologically compatible materials, and release thereof in active form. In a particular aspect, the present invention relates to novel constructs containing one or more biologically compatible materials reversibly linked thereto. In a further aspect, the invention relates to methods for delivering biologically compatible materials to a subject in need thereof. In a still further aspect, the invention relates to a method for modifying biologically compatible materials to enhance the transport and/or bioavailability thereof.

### BACKGROUND OF THE INVENTION

The potential use of charged molecules such as polynucleotides as therapeutic agents has attracted great attention as a novel approach for treating severe and chronic diseases. However, charged molecules such as polynucleotides have poor bioavailability and uptake into cells because such molecules do not readily permeate the cellular membrane due to the charge repulsion between the negatively charged membrane and the high negative charge on the molecule to be delivered. In addition, charged molecules such as polynucleotides are also highly susceptible to rapid nuclease degradation both inside and outside the cytoplasm; see examples from Geary et al, J. Pharmacol. Exp. Ther. 296:890-897 (2001).

One strategy to improve the structural stability of polynucleotides in vivo is to modify the phosphodiester backbone structure of the polynucleotides in efforts to reduce enzymatic susceptibility. Other strategies for addressing stability and delivery of polynucleotides include condensation of cationic molecules (such as viral vectors) with polynucleotides and cationic delivery system (such as lipid vesicles, lipid nanoparticles, polyethyleneimines and cyclodextrin-based polymers). However, concerns with intracellular vehicle fate and toxicity remain high. There is an ongoing need for improved compositions and methods for binding, stabilization and cellular delivery of charged molecules and for therapeutic treatment of diseases using same.

### SUMMARY OF THE INVENTION

In accordance with the present invention, novel multifunctional compounds have been developed which have orthogonal reactive groups thereon, thereby facilitating preparation of compounds having multiple functional properties (e.g., a targeting moiety and a biologically active moiety). Such constructs are useful for a variety of applications, e.g., for the delivery of biologically compatible materials, and release thereof in active form.

Therefore, in accordance with the present invention, there are provided multifunctional linkers of defined structure, as well as various derivatives thereof bearing one or more biologically active components thereon.

Also provided in accordance with the present invention are methods for the preparation of such constructs, as well as various uses thereof.

Invention constructs are useful for a variety of applications, e.g., such constructs can facilitate delivery of a biologically active moiety (e.g., siRNA) to a target destination, and release of the active moiety therefrom (or elimination of protective agents or coating agents therefrom) upon arrival at the targeted cells/tissues/organs.

### BRIEF DESCRIPTION OF THE FIGURES

FIG. 1 illustrates the effect of particle size on the rate of PEG hydrolysis at pH 5. See, for example, Example 17.

FIG. 2 illustrates the time-course of salt induced particle aggregation for several compounds according to the present invention. See, for example, Example 17.

### DETAILED DESCRIPTION OF THE INVENTION

In accordance with the present invention, there are provided multifunctional linkers having the structure:

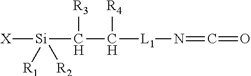

wherein:
X is a leaving group selected from the group consisting of —Cl, —Br, —I and —OSO₂R, wherein R is an optionally substituted lower alkyl, an optionally substituted aryl, or an optionally substituted heteroaryl;
R₁ and R₂ are independently optionally substituted lower alkyl, optionally substituted aryl, or optionally substituted heteroaryl;
R₃ and R₄ are independently hydrogen or optionally substituted lower alkyl, or, R₃ and R₄, taken together, are C₁-C₅ alkylene or substituted alkylene; and
L₁ is a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear spacer element, and combinations of any two or more thereof, or R₃ and L₁, or R₄ and L₁, taken together, are C₁-C₅ alkylene or substituted alkylene;
provided, however, when R₃ and R₄ are hydrogen, L₁ is a covalent bond, methylene, ethylene, —CH₂C(═O)—, or —(CH₂)₈C(═O)—, and X is chloro, at least one of R₁ and R₂ is not methyl.

In accordance with another aspect of the present invention, there are provided constructs obtained by displacing X of the above-described linker with alcohol A-OH or carboxylic acid A-CO₂H, thereby producing the structure:

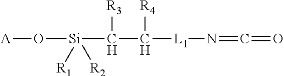

wherein:
A is an organic moiety containing at least 5 up to about 100 carbon atoms, excluding alcohols A-OH selected from the group consisting of benzyl alcohol, 2-methylphenol, 3-methylphenol, and 1-octadecanol.

In certain aspects, organic moiety, A, is selected such that the resulting alcohol A-OH or carboxylic acid A-CO₂H employed in the practice of the present invention has its own, known biological activity or therapeutic activity.

Exemplary alcohols (A-OH) or carboxylic acids (A-CO₂H) contemplated for use herein typically have at least 5 carbon atoms, and up to about 100 carbon atoms. In some embodiments, organic moiety, A, has 5-80 carbon atoms; in other embodiments, organic moiety, A, has 6-60 carbon atoms; in still other embodiments, organic moiety, A, has 6-40 carbon atoms; in yet other embodiments, organic moiety, A, has 6-30 carbon atoms. Exemplary alcohols and acids are selected from the group consisting of polyethylene glycol, a polyethylene glycol derivative, a sterol, a sterol derivative, cholic acid, a cholic acid derivative, a secosteroid, a monoglyceride, a diglyceride, a phospholipid, a phospholipid derivative, a fatty acid, a fatty acid derivative, sirolimus, everolimus, paclitaxel, docetaxel, camptothecin, epothilone, an epothilone derivative, doxorubicin, a maytansinoid, an ansamycin, a retinoid, a tocopherol, a tocotrienol, biotin, a vitamin B compound, duocarmycin, a duocarmycin derivative, auristatin, an auristatin derivative, and the like.

As used herein, “alkyl” refers to saturated straight or branched chain hydrocarbon radical having in the range of 1 up to about 20 carbon atoms. “Substituted alkyl” refers to alkyl groups further bearing one or more substituents selected from alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrone, amino, amido, —C(O)H, acyl, oxyacyl, carboxyl, carbamate, dithiocarbamoyl, sulfonyl, sulfonamide, sulfuryl, and the like.

As used herein, “halogen” refer to all halogens, that is, chloro (Cl), fluoro (F), bromo (Br), or iodo (I). In certain embodiments, “halogen” refers specifically to the subset of halogens comprising —Cl, —Br or —I.

As used herein, “lower alkyl” refers to an alkane-derived group containing from 1 to 6 carbon atoms (unless specifically defined) that includes a straight chain alkyl or branched alkyl. The straight chain or branched lower alkyl group is chemically feasible and attached at any available point to provide a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. “Substituted lower alkyl” refers to lower alkyl independently substituted as described herein, unless indicated otherwise, with one or more, preferably 1, 2, 3, 4 or 5, also 1, 2, or 3 substituents, wherein the substituents are as indicated. For example “fluoro substituted lower alkyl” denotes a lower alkyl group substituted with one or more fluoro atoms, such as perfluoroalkyl, where preferably the lower alkyl is substituted with 1, 2, 3, 4 or 5 fluoro atoms, also 1, 2, or 3 fluoro atoms. It is understood that any such substitutions, or substitution of lower alkyl on another moiety, are chemically feasible and attached at any available atom to provide a stable compound.

As used herein, alkylene refers to saturated, divalent straight or branched chain hydrocarbyl groups typically having in the range of about 2 up to about 12 carbon atoms, and “substituted alkylene” refers to alkylene groups further bearing one or more substituents as set forth above.

As used herein, “alkenyl” refers to straight or branched hydrocarbons containing 2-12 carbon atoms (unless specifically defined) and at least one, preferably 1-3, more preferably 1-2, most preferably one, carbon to carbon double bond. Carbon to carbon double bonds may be either contained within a straight chain or branched portion. The straight chain or branched alkenyl group is chemically feasible and attached at any available point to provide a stable compound. Examples of alkenyl groups include ethenyl, propenyl, isopropenyl, butenyl, and the like. A “substituted alkenyl” denotes alkenyl that is independently substituted, unless indicated otherwise, with one or more, preferably 1, 2, 3, 4 or 5, also 1, 2, or 3 substituents, wherein the substituents are as indicated. It is understood that any such substitutions, or substitution of alkenyl on another moiety, are chemically feasible and attached at any available atom to provide a stable compound.

As used herein, “alkynyl” refers to a straight or branched hydrocarbon containing 2-12 carbon atoms (unless specifically defined) containing at least one, preferably one, carbon to carbon triple bond. The straight chain or branched alkynyl group is chemically feasible and attached at any available point to provide a stable compound. Examples of alkynyl groups include ethynyl, propynyl, butynyl, and the like. A “substituted alkynyl” denotes alkynyl that is independently substituted, unless indicated otherwise, with one or more, preferably 1, 2, 3, 4 or 5, also 1, 2, or 3 substituents, wherein the substituents are as indicated. It is understood that any such substitutions, or substitution of alkynyl on another moiety, are chemically feasible and attached at any available atom to provide a stable compound.

As used herein, “cycloalkyl” refers to saturated or unsaturated, non-aromatic monocyclic carbon ring systems of 3-10, also 3-8, more preferably 3-6, ring members per ring, such as cyclopropyl, cyclopentyl, cyclohexyl, cycloheptyl, and the like. A “substituted cycloalkyl” is a cycloalkyl that is independently substituted, unless indicated otherwise, with one or more, preferably 1, 2, 3, 4 or 5, also 1, 2, or 3 substituents, wherein the substituents are as indicated. It is understood that substitutions on cycloalkyl, or substitution of cycloalkyl on another moiety, are chemically feasible and attached at any available atom to provide a stable compound.

As used herein, “aryl” refers to a monocyclic or bicyclic ring system containing aromatic hydrocarbons such as phenyl or naphthyl, which may be optionally fused with a cycloalkyl of preferably 5-7, more preferably 5-6, ring members. A “substituted aryl” is an aryl that is independently substituted, unless indicated otherwise, with one or more, preferably 1, 2, 3, 4 or 5, also 1, 2, or 3 substituents, wherein the substituents are as indicated. It is understood that substitutions on aryl, or substitution of aryl on another moiety, are chemically feasible and attached at any available atom to provide a stable compound.

As used herein, “heteroaryl” refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is provided. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A “substituted heteroaryl” is a heteroaryl that is independently substituted, unless indicated otherwise, with one or more, preferably 1, 2, 3, 4 or 5, also 1, 2, or 3 substituents, wherein the substituents are as indicated. It is understood that substitutions on heteroaryl, or substitution of heteroaryl on another moiety, are chemically feasible and attached at any available atom to provide a stable compound.

As used herein, “heteroalkylene” refers to refers to saturated, divalent straight or branched chain hydrocarbyl groups typically having in the range of about 2 up to about 12 carbon atoms, and one or more heteroatoms (e.g., N, S, or O) in the backbone thereof “Substituted heteroalkylene” refers to heteroalkylene groups further bearing one or more substituents as set forth above.

As used herein, “alkenylene” refers to divalent straight or branched chain hydrocarbyl groups having at least one carbon-carbon double bond, and typically having in the range of about 2 up to 12 carbon atoms, and “substituted alkenylene” refers to alkenylene groups further bearing one or more substituents as set forth above.

As used herein, “heteroalkenylene” refers to divalent straight or branched chain hydrocarbyl groups having at least one carbon-carbon double bond, and typically having in the range of about 2 up to 12 carbon atoms, and one or more heteroatoms (e.g., N, S or O) in the backbone thereof “Substituted heteroalkenylene” refers to heteroalkenylene groups further bearing one or more substituents as set forth above.

As used herein, “alkynylene” refers to divalent straight or branched chain hydrocarbyl groups having at least one carbon-carbon triple bond, and typically having in the range of about 2 up to 12 carbon atoms, and “substituted alkynylene” refers to alkynylene groups further bearing one or more substituents as set forth above.

As used herein, “heteroalkynylene” refers to divalent straight or branched chain hydrocarbyl groups having at least one carbon-carbon triple bond, and typically having in the range of about 2 up to 12 carbon atoms, and one or more heteroatoms (e.g., N, S or O) in the backbone thereof “Substituted heteroalkynylene” refers to heteroalkynylene groups further bearing one or more substituents as set forth above.

As used herein, “arylene” refers to divalent aromatic groups typically having in the range of 6 up to 14 carbon atoms and “substituted arylene” refers to arylene groups further bearing one or more substituents as set forth above.

As used herein, “heteroarylene” refers to divalent aromatic groups typically having in the range of 6 up to 14 carbon atoms and one or more heteroatoms (e.g., N, S or O) as an integral part of the ring. “Substituted heteroarylene” refers to heteroarylene groups further bearing one or more substituents as set forth above.

As used herein, “cyloalkylene” refers to divalent saturated or unsaturated, non-aromatic monocyclic carbon ring systems of 3-10, also 3-8, more preferably 3-6, ring members per ring, such as cyclopropylene, cyclopentylene, cyclohexylene, cycloheptylene, and the like. A “substituted cycloalkylene” is a cycloalkylene that is independently substituted, unless indicated otherwise, with one or more, preferably 1, 2, 3, 4 or 5, also 1, 2, or 3 substituents, wherein the substituents are as indicated. It is understood that substitutions on cycloalkylene, or substitution of cycloalkylene on another moiety, are chemically feasible and attached at any available atom to provide a stable compound.

As used herein, “heterocyloalkylene” refers to divalent saturated or unsaturated, non-aromatic monocyclic carbon ring systems of 3-10, also 3-8, more preferably 3-6, ring members per ring, containing one or more heteroatoms (e.g., N, S or O) as an integral part of the ring structure. A “substituted heterocycloalkylene” is a heterocycloalkylene that is independently substituted, unless indicated otherwise, with one or more, preferably 1, 2, 3, 4 or 5, also 1, 2, or 3 substituents, wherein the substituents are as indicated. It is understood that substitutions on heterocycloalkylene, or substitution of heterocycloalkylene on another moiety, are chemically feasible and attached at any available atom to provide a stable compound.

In certain embodiments of the present invention, linker L₁ is selected from the group consisting of a covalent bond, C₁-C₆ alkylene, substituted C₁-C₆ alkylene, C₂-C₆ alkenylene, substituted C₂-C₆ alkenylene, arylene, substituted arylene, heteroarylene, substituted heteroarylene, C₃-C₇ cyloalkylene, or substituted C₃-C₇ cycloalkylene, and the like, as well as combinations of any two or more thereof.

As used herein, a linear spacer element refers to a substantially linear moiety with at least two attachment sites separated by a distance in the range of about 5-35 Angstroms. Exemplary linear spacer elements include substituted biphenyls (10 Angstrom distance between anchor points (A,B) at the para-positions), substituted biphenyl ethers (10 Angstrom distance between anchor points at the para-positions), bilirubin (15 Angstrom distance between anchor points for arms) and octaphenyl (35 Angstrom distance between anchor points for arms) as illustrated below:

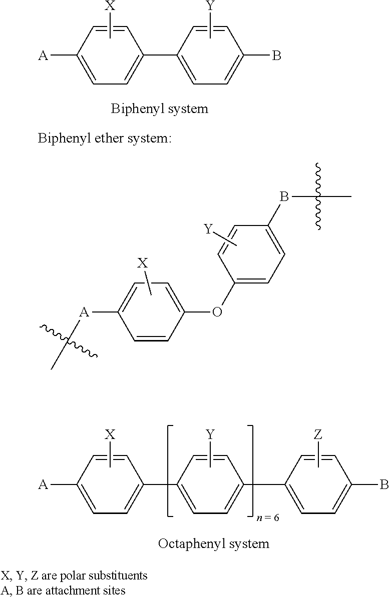

In certain embodiments of the present invention, R₁ is methyl, ethyl, n-propyl, isopropyl, n-butyl, isobutyl, t-butyl, or the like; R₂ is methyl, ethyl, n-propyl, isopropyl, n-butyl, isobutyl, t-butyl, or the like; R₃ is hydrogen, methyl, ethyl, or the like; and R₄ is hydrogen, methyl, ethyl, or the like.

In some embodiments of the present invention, R₁ is methyl, ethyl, n-propyl, isopropyl, n-butyl, isobutyl, t-butyl, or the like; R₂ is methyl, ethyl, n-propyl, isopropyl, n-butyl, isobutyl, t-butyl, or the like; and R₃ and R₄ cooperate to form a C₃-C₇ cycloalkylene or substituted C₃-C₇ cycloalkylene ring.

In certain embodiments of the present invention, R₁ is methyl, ethyl, n-propyl, isopropyl, n-butyl, isobutyl, t-butyl, or the like; R₂ is methyl, ethyl, n-propyl, isopropyl, n-butyl, isobutyl, t-butyl, or the like; and R₃ and L₁ cooperate to form a C₄-C₇ cycloalkylene or substituted C₄-C₇ cycloalkylene ring.

In some embodiments of the present invention, R₁ is methyl, ethyl, n-propyl, isopropyl, n-butyl, t-butyl, or the like; R₂ is methyl, ethyl, n-propyl, isopropyl, n-butyl, t-butyl, or the like; and R₄ and L₁ cooperate to form a C₃-C₇ cycloalkylene or substituted cycloalkylene ring.

In accordance with another aspect of the present invention [[[claim 10]]], there are provided constructs having the structure:

A-L₂-B—Y—C-L₃-Z

wherein:
A is a biologically compatible material containing at least 4 carbon atoms;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from.

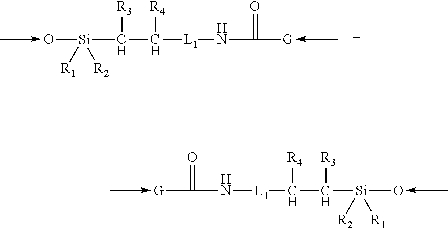

wherein:
R₁ and R₂ are independently optionally substituted lower alkyl, optionally substituted aryl, or optionally substituted heteroaryl;
R₃ and R₄ are independently hydrogen or optionally substituted lower alkyl, or, R₃ and R₄, taken together, are C₁-C₅ alkylene or substituted alkylene;
L₁ is a covalent bond, or a linker selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof, or R₃ and L₁, or R₄ and L₁, taken together, are C₁-C₅ alkylene or substituted alkylene;
G is selected from O, N, S; and
Z is a reactive group selected from the group consisting of thiols, disulfides, esters, thioesters, amines, anhydrides, hydrazines, aldehydes, ketones, boronic acids, carboxylic acids, azides, alkyl halides, alkenes, alkynes, alcohols, isocyanates, isothiocyanates, sulfonyl chlorides, epoxides, carbonates, hydroxymethyl phosphines, 2-iminothiolanes, and aziridines.

In accordance with yet another aspect of the present invention [[[Claim 11]]], there are provided constructs having the structure:

A-L₂-B—Y—C-L₃-Z

wherein:
A is a biologically compatible material;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core

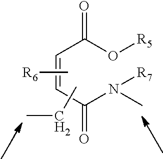

wherein:
R₅ is optionally present, and, when present, is selected from H, an alkali metal ion, an ammonium ion;
R₆ and R₇ are independently hydrogen, optionally substituted lower alkyl, optionally substituted aryl, or optionally substituted heteroaryl; and
Z is a reactive group selected from the group consisting of thiols, —SSR(R=aryl, heteroaryl), amines, azides, alkynes. alkenes.

In accordance with still another aspect of the present invention [[[Claim 12]]], there are provided constructs having the structure:

A-L₂-B—Y—C-L₃-Z

wherein:
A is a biologically compatible material;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from:

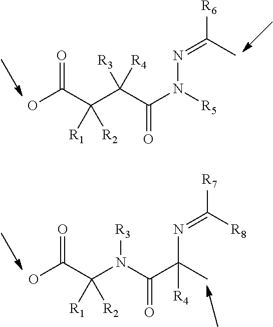

wherein:
R₁, R₂, R₃ and R₄ are independently hydrogen, optionally substituted lower alkyl, optionally substituted aryl, or optionally substituted heteroaryl; or, R₁ and R₃, taken together, are C₁-C₆ alkylene or substituted alkylene;
R₅ is optionally substituted lower alkyl;
R₆ is optionally substituted lower alkyl, optionally substituted aryl, or optionally substituted heteroaryl;
R₇ and R₈ are independently optionally substituted aryl, or optionally substituted heteroaryl, provided, however, that one of R₇ and R₈ is ortho-alkoxy substituted aryl; and
Z is selected from the group consisting of thiols, disulfides, esters, thioesters, amines, anhydrides, hydrazines, aldehydes, ketones, boronic acids, carboxylic acids, azides, alkyl halides, alkenes, alkynes, alcohols, isocyanates, isothiocyanates, sulfonyl chlorides, epoxides, carbonates, hydroxymethyl phosphines, 2-iminothiolanes, and aziridines.

A wide variety of biologically compatible materials, A, are contemplated for use herein. Exemplary materials include biologically active molecules, bio-recognition molecules, peptides, oligopeptides, proteins, lipids, oligosaccharides, nucleic acids, polyethylene glycols, macrocycles, oligonucleotides, and the like.

Biologically active molecules contemplated for use herein include physiologically active molecules, antibodies, natural products, small molecule drugs, and the like.

A wide variety of bio-recognition molecules are contemplated for incorporation into invention molecular entities, e.g., oligopeptides or oligosaccharides that are involved in a large range of biological processes that promote binding or recognition of such oligopeptides or oligosaccharides (examples of such peptidyl-cyclodextrins can be found in Pean et al. J. Chem. Soc. Perkin Trans. 2, 2000, 853-863), antibodies, cell targeting motifs, cell penetrating motifs, membrane active peptides (e.g., fusogenic peptide sequences, endosomolytic peptide sequences, and the like), and the like.

As used herein, the term “cell targeting motifs” embraces a peptide sequence, an epitope on a peptide, or a chemical subunit which has affinity to a specific site, location, or recognition site on the surface of a cell without necessarily causing internalization (see, for example, Biochemical Society Transaction (2007) Vol. 35, 780-783).

As used herein, the term “cell penetrating motifs” embraces a peptide sequence, an epitope on a peptide, or a chemical subunit that translocates the cell membrane and facilitates the transport of various molecular cargo across the cell membrane.

As used herein, the term “membrane active peptides” embraces peptides capable of interacting with and/or destabilizing membrane bilayers. Examples of such peptides include fusogenic peptides, endosomolytic peptides, and the like.

As used herein, “peptide” refers to a compound comprising two or more amino acids linked covalently through a peptide bond (i.e., the bond between the alpha-carboxyl group of one amino acid and the alpha-amino group of the next be elimination of a molecule of water).

As used herein, “oligopeptides” are peptides comprising in the range of about 3 up to about 15 amino acids; preferably in the range of about 3 up to about 10 amino acids.

As used herein, “polypeptides” are peptides comprising a plurality of amino acids; typically at least 15 or more amino acids, with polypeptides preferably comprising 20 or more amino acids.

As used herein, “protein complexes” are polypeptides comprising more than one polypeptide chain.

As used herein, “oligosaccharides” refer to a carbohydrate containing 3 or more monosaccharide units.

As used herein, the term “nucleic acids” are oligonucleotides consisting of deoxyribonucleic acid (DNA) or ribonucleic acid (RNA), or chimeric oligonucleotides, containing DNA and RNA, or oligonucleotide strands containing non-natural monomers, including but not limited to 2′-methoxy or 2′-fluoro-modified nucleotides with ribo- or arabino-stereochemistry at the 2′-position, or thio-substituted phosphate groups. Nucleic acids contemplated for use in the practice of the present invention may also include conjugated nucleic acids where nucleic acids conjugate to protein, polypeptide or any organic molecules.

A wide variety of polyethylene glycols (PEG's) can be employed in the practice of the present invention, including branched or linear PEG's, and PEG's having a wide range of molecular weights; with molecular weights in the range of about 500 up to about 25,000 being presently preferred.

In certain aspects of the present invention, the PEG employed for preparation of pegylated molecular entities may optionally contain one or more peptide segments which are susceptible to enzymatic cleavage.

When invention molecular entities are pegylated, PEG can be incorporated into the molecular entity in a variety of ways, e.g., via a disulfide linkage, a thioether linkage, an ester linkage, an amide linkage, a maleimide linkage, a thio-maleimide linkage, a sulfone linkage, a carbamate linkage, an urea linkage, and the like.

As used herein, “macrocycle” refers to large ring structures such as cyclic peptides, cyclic oligosaccharides (e.g. cyclodextrins), cyclic oligoethyleneglycols, substituted porphyrins, substituted corrins, substituted corroles, and the like.

As used herein, “oligonucleotide” refers to a sequence of two or more deoxyribonucleotides, ribonucleotides or analogs thereof that are linked together by a phosphodiester bond or other known linkages. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. The terms are also used herein to include naturally occurring nucleic acid molecules, which can be isolated from a cell using recombinant DNA methods, as well as synthetic molecules, which can be prepared, for example, by methods of chemical synthesis or by enzymatic methods such as by PCR. The term “recombinant” is used herein to refer to a nucleic acid molecule that is manipulated outside of a cell, including, for example, a polynucleotide encoding an siRNA specific for a histone H4 gene operatively linked to a promoter. Preferred length of oligonucleotides in double-stranded nucleic acids is between 15-60 monomers; more preferred length is between 15-45 monomers; even more preferred length is between 19-30 monomers; most preferred length is between 21-27 monomers.

In accordance with some aspects of the present invention, the hydrolytically labile core Y of constructs having the structure A-L₂-B—Y—C-L₃-Z is cleavable under physiological conditions. Such conditions include

pH:
in the range of about 7.0 up to about 7.4 (or higher if a cell is growing), or
about 6.5 in cancer cells, or
in the range of about 5-6 in endosomes, or
in the range of about 5-5.5 in lysosomes;

Temperature of about 37° C.,

Glucose concentration of about 0.5 mM,

Glutathione concentration in the range of about 1 up to about 10 mM
(increased levels are observed in tumor cells (e.g., colorectal cancer, breast cancer, and the like), relative to surrounding tissue).

Ion concentration is typically about 150 mM, comprising:

| | Concentration in | Concentration |
|---|---|---|
| Ion | cytosol (mM) | in blood (mM) |
| Potassium | 139 | 4 |
| Sodium | 12 | 145 |
| Chloride | 4 | 116 |
| Bicarbonate | 12 | 29 |
| AA in proteins | 138 | 9 |
| Magnesium | 0.8 | 1.5 |
| Calcium | <0.0002 | 1.8. |

In accordance with some aspects of the present invention, the hydrolytically labile core Y of constructs having the structure A-L2-B—Y—C-L3-Z is cleavable under conditions existing in certain intracellular compartments, in malignant cells, in foreign cells (e.g., parasites), in cells undergoing specific changes related to disease states (e.g., inflammation, apoptosis, starvation, and the like), and the like. Such conditions can differ from physiological conditions in a variety of ways, e.g., in redox potential (cytoplasm), pH (endosome, lysosome), temperature, salt concentration, concentration of catalytically active proteins and/or macromolecules (e.g., heparin, RNA, DNA), and the like.

In accordance with certain aspects of the present invention, L₂ and L₃ of constructs having the structure A-L₂-B—Y—C-L₃-Z are each independently selected from the group consisting of a covalent bond, alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof.

In accordance with some aspects of the present invention, the reactive group Z of constructs having the structure A-L₂-B—Y—C-L₃-Z is selected from the group consisting of thiols, disulfides, esters, thioesters, amines, anhydrides, hydrazines, aldehydes, ketones, boronic acids, azides, alkyl halides, alkenes, alkynes, alcohols, isocyanates, isothiocyanates, sulfonyl chlorides, epoxides, carbonates, hydroxymethyl phosphines, 2-iminothiolanes, aziridines, and the like.

In accordance with yet another aspect of the present invention, there are provided compositions comprising any of the constructs described herein, and a pharmaceutically acceptable carrier therefore.

In accordance with certain embodiments of the present invention, there are provided compositions comprising the construct:

A-L₂-B—Y—C-L₃-Z

and a pharmaceutically acceptable carrier therefore. Such compositions may optionally further comprise the construct D-X″, wherein:
D of the construct D-X″ is a biologically compatible material, and
X″ is a reactive group which is reactive with said reactive group Z.

The phrase “pharmaceutically acceptable carrier” refers to any carrier known to those skilled in the art to be suitable for the particular mode of administration. In addition, invention compounds and constructs may be formulated as the sole pharmaceutically active ingredient in the composition or may be combined with other active ingredients.

Compositions herein comprise one or more compounds and/or constructs provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds and/or constructs described herein are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof or constructs containing same is (are) mixed with a suitable pharmaceutical carrier. The compounds and/or constructs containing same may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds and/or constructs in the compositions are effective for delivery of an amount, upon administration, that treats, prevents, or ameliorates one or more of the symptoms of diseases or disorders to be treated.

The phrase “pharmaceutically acceptable salt” refers to any salt preparation that is appropriate for use in a pharmaceutical application. Pharmaceutically-acceptable salts include amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane, and the like; alkali metal salts, such as lithium, potassium, sodium, and the like; alkali earth metal salts, such as barium, calcium, magnesium, and the like; transition metal salts, such as zinc, aluminum, and the like; other metal salts, such as sodium hydrogen phosphate, disodium phosphate, and the like; mineral acids, such as hydrochlorides, sulfates, and the like; and salts of organic acids, such as acetates, lactates, malates, tartrates, citrates, ascorbates, succinates, butyrates, valerates, fumarates, and the like.

In one embodiment, the compositions and/or constructs are formulated for single dosage administration. To formulate a composition, the weight fraction of compound and/or construct containing same is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and in PCT publication WO 04/018997, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% (wt %) with the balance made up from non-toxic carrier may be prepared. Methods for preparation of these compositions are known to those skilled in the art. The contemplated compositions may contain 0.001%-100% (wt %) active ingredient, in one embodiment 0.1-95% (wt %), in another embodiment 75-85% (wt %).

Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% (vol %) isotonic solutions, pH about 5-7, with appropriate salts.

Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### Therapeutic Applications

The invention contemplates administration of the above-described compounds, constructs and pharmaceutical compositions to subjects. As used herein, the term “subject” includes humans, as well as laboratory animals, veterinary animals, and animals of commercial interest, e.g., bovine, ovine, and the like.

Compounds and compositions of the instant invention may be used to treat or ameliorate a variety of disorders. Compounds and compositions that may be used in therapeutic applications, in one embodiment have reasonably high bioavailability in a target tissue (i.e. brain, for neurodegenerative disorders; particular peripheral organs for other conditions), and reasonably low toxicity. Those skilled in the art can assess compounds and compositions described herein for their pharmaceutical acceptability using standard methods.

For instance, compounds and compositions of the instant invention can be used in the treatment of cancer or other diseases characterized by abnormal cellular proliferation, inflammatory disease, bacterial or viral infection, autoimmune disease, acute pain, muscle pain, neuropathic pain, allergies, neurological disease, dermatological conditions, cardiovascular disease, diabetes, gastrointestinal disorders, depression, endocrine or other disease characterized by abnormal hormonal metabolism, obesity, osteoporosis or other bone disorders, pancreatic disease, epilepsy or seizure disorders, erectile or sexual dysfunction, opthamological disorders or diseases of the eye, cholesterol imbalance, hypertension or hypotension, migraine or headaches, obsessive compulsive disorder, panic disorder, anxiety disorder, post traumatic stress disorder, chemical dependency or addiction, and the like.

Those skilled in the art can determine other diseases and disorders for which administration of a compound or composition described herein can be beneficial.

In accordance with still another embodiment of the present invention, there are provided methods for protecting biologically active materials, said method comprising reacting a biologically active material, A, with the above-described linker, thereby appending said biologically active material to one of the reactive moieties of said linker (i.e., at “X” or at the —N═C═O moiety thereof). Before, or after, introduction of A, the other reactive moiety of said linker can be reacted with a targeting agent, a stabilizing agent, a membrane active agent, a catalytically active agent, or the like.

The resulting modified form of biologically active material A can then be administered to a subject in need thereof, whereby the biologically active material will be targeted to the desired site to facilitate treatment and/or the biologically active material will be protected from the effect of physiological conditions until it has been transported to the desired site.

In accordance with still another aspect of the present invention [[[claim 19]]], there are provided constructs having the structure:

A-L₂-B—Y—C-L₄-D

wherein:
A and D are independently biologically compatible materials containing at least 4 carbon atoms;
B and C are independently a covalent bond or a methylene unit optionally mono-substituted or di-substituted with lower alkyl, optionally substituted aryl, or optionally substituted heteroaryl;
L₂ and L₄ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof;
Y is a hydrolytically labile core selected from:

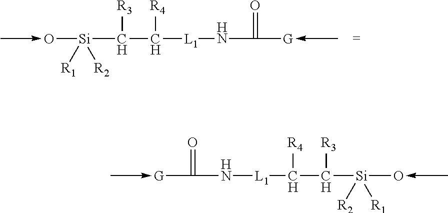

wherein:
R₁ and R₂ are independently optionally substituted lower alkyl, optionally substituted aryl, or optionally substituted heteroaryl;
R₃ and R₄ are independently hydrogen, optionally substituted lower alkyl, or R₃ and R₄, taken together, are C₁-C₅ alkylene or substituted alkylene;
L₁ is a covalent bond, or a linker selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof, or R₃ and L₁, or R₄ and L₁, taken together, are C₁-C₅ alkylene or substituted alkylene; and
G is selected from O, N, S.

In accordance with still another aspect of the present invention [[[claim 20]]], there are provided constructs having the structure:

A-L₂-B—Y—C-L₄-D

wherein:
A and D are independently biologically compatible materials;
B and C are independently a covalent bond or a methylene unit optionally mono-substituted or di-substituted with lower alkyl, optionally substituted aryl, or optionally substituted heteroaryl;
L₂ and L₄ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from:

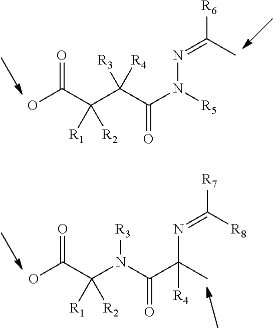

wherein:
R₁, R₂, R₃ and R₄ are independently hydrogen, optionally substituted lower alkyl, optionally substituted aryl, or optionally substituted heteroaryl; or, R₁ and R₃, taken together, are C₁-C₆ alkylene or substituted alkylene;
R₅ is optionally substituted lower alkyl;
R₆ is optionally substituted lower alkyl, optionally substituted aryl, or optionally substituted heteroaryl; and
R₇ and R₈ are independently optionally substituted aryl, or optionally substituted heteroaryl, provided, however, that one of R₇ and R₈ is ortho-alkoxy substituted aryl.

In accordance with certain aspects of the present invention, biologically compatible materials A and D of the construct A-L₂-B—Y—C-L₄-D are independently selected from the group consisting of biologically active molecules, peptides, oligopeptides, polypeptides, proteins, protein complexes, antibodies, oligosaccharides, nucleic acids, polyethylene glycols, amphiphilic macrocycles, oligonucleotides, and the like.

In accordance with some aspects of the present invention, the hydrolytically labile core Y of constructs having the structure A-L₂-B—Y—C-L₄-D is cleavable under physiological conditions. In certain aspects, the hydrolytically labile core Y is cleavable under conditions existing in certain intracellular compartments, in malignant cells, in foreign cells (e.g., parasites), in cells undergoing specific changes related to disease states (e.g., inflammation, apoptosis, starvation, and the like), and the like.

In accordance with certain aspects of the invention, L₄ of the construct A-L₂-B—Y—C-L₄-D is produced by the reaction of E of the construct D-E with Z of the construct A-L₂-B—Y—C-L₃-Z, wherein E is a reactive group which reacts with Z. The conditions under which the reaction between A-L₂-B—Y—C-L₃-Z and D-E can take place comprise dissolving the reactants in a suitable polar solvent (e.g., dimethylsulfoxide, dimethylformamide, dimethylacetamide, an aqueous solution, or the like, and/or mixtures thereof) and subjecting same to temperatures between about 0° and about 40° Celsius at a pH ranging from about 6 to about 9.

Exemplary reactive groups E and Z include thiols, disulfides, esters, thioesters, amines, anhydrides, hydrazines, aldehydes, ketones, boronic acids, azides, alkyl halides, alkenes, alkynes, alcohols, isocyanates, isothiocyanates, sulfonyl chlorides, epoxides, carbonates, hydroxymethyl phosphines, 2-iminothiolanes, aziridines, and the like.

In accordance with certain aspects of the present invention, the linkage between L₄ and D in constructs having the structure A-L₂-B—Y—C-L₄-D results from the reaction of E (of D-E) with Z (of A-L₂-B—Y—C-L₃-Z) under physiological conditions. Exemplary linkages between L₄ and D include a disulfide, an amide, a triazole, a urea, a thiourea, a thioether, a sulfonamide, a boronate, an amine, an amidine, a carbamate, a guanidine, an imine, a hydrazone, and the like.

In certain embodiments, the linkage between L₄ and D in constructs having the structure A-L₂-B—Y—C-L₄-D is a disulfide, a silyl ether, a hydrazone, a ketal, an acetal, a maleate amide, a boronate, an imine, or the like.

In some embodiments of the present invention, the linkage between L₄ and D in constructs having the structure A-L₂-B—Y—C-L₄-D is cleavable under physiological conditions.

In accordance with still another embodiment of the present invention, there are provided compositions comprising the construct:

A-L₂-B—Y—C-L₄-D

and a pharmaceutically acceptable carrier therefore.

In accordance with yet another aspect of the present invention, there are provided methods for delivering a biologically compatible material to a subject in need thereof, said method comprising administering to said subject an effective amount of the construct A-L₂-B—Y—C-L₄-D.

In certain aspects, invention methods of delivering a biologically compatible material A, or derivative thereof, to a subject in need thereof, comprise administering to said subject an effective amount of a composition comprising a construct having the structure A-L₂-B—Y—C-L₄-D, wherein Y is further characterized by cleaving under physiological conditions to produce constructs containing A-L₂ and/or D-L₄ as part thereof.

In some aspects, there are provided methods of delivering a biologically compatible material A, or derivative thereof, to a subject in need thereof, said methods comprising administering to said subject an effective amount of a combination comprising a construct having the structure A-L₂-B—Y—C-L₃-Z and the construct D-E, wherein:
E is a reactive group characterized by reacting with Z to form a linkage between L₄ and D, and
D is a biologically compatible material.

In other aspects of the present invention, there are provided methods of delivering a biologically compatible material D, or derivative thereof, to a subject in need thereof, said method comprising administering to said subject an effective amount of a combination comprising a construct having the structure A-L₂-B—Y—C-L₃-Z and the construct D-E, wherein:
E is a reactive group characterized by:
   reacting with Z under physiological conditions to form a linkage between L₄ and D, or
   forming a covalent bond between L₄ and D which bond is cleavable under physiological conditions, and
D is a biologically compatible material.

In accordance with a still further aspect of the present invention, there are provided methods for modifying a biologically compatible material A with a modifying agent D, said method comprising contacting the construct A-L₂-B—Y—C-L₃-Z with the construct D-E under conditions suitable for the formation of the construct A-L₂-B—Y—C-L₄-D.

In accordance with yet another aspect of the present invention, there are provided methods of modifying a biologically compatible material D with a modifying agent A, said method comprising contacting the construct A-L₂-B—Y—C-L₃-Z with the construct D-E under conditions suitable for the formation of the construct A-L₂-B—Y—C-L₄-D.

In accordance with another aspect of the present invention, there are provided methods for preparing the construct A-L₂-B—Y—C-L₄-D, said method comprising contacting A-L₂-B—Y—C-L₃-Z with D-E under conditions suitable for the formation of the construct A-L₂-B—Y—C-L₄-D.

Also provided in accordance with the present invention are methods for releasing active component A from the construct A-L₂-B—Y—C-L₄-D, said method comprising subjecting said construct to physiological conditions suitable to cleave the hydrolytically labile core, Y, or the bond adjacent to at least one of the L₂ or the L₄ linkages.

In accordance with yet another aspect of the present invention, there are provided methods for releasing active component D from the construct A-L₂-B—Y—C-L₄-D, said methods comprising subjecting said construct to physiological conditions suitable to cleave the hydrolytically labile core, Y, or the bond adjacent to at least one of the L₃ or the L₄ linkages.

The invention will now be described in greater detail with reference to the following non-limiting examples.

### EXAMPLES

Abbreviations used throughout the examples are defined as follows:
DCM=dichloromethane,
DIC=diisopropyl carbodiimide,
DIEA=diisopropyl ethylamine,
DMAP=dimethyl amino pyridine,
DME=1,2-dimethoxyethane,
DMF=dimethyl formamide,
DMSO=dimethyl sulfoxide,
EDTA=ethylene diamine tetraacetic acid,
HEPES=4-(2-hydroxyethyl)-1-piperazineethanesulfonic acid,
HOBt=1-hydroxybenzotriazole,
PEG=polyethylene glycol,
Py=pyridine,
TCPC=Tailored Cyclodextrin Peptide Conjugates with the peptides sequences containing one or more lysine and one or more cysteine residues (see, for example PCT/US2009/052899), and
TFA=trifluoroacetic acid.

### Example 1

### Preparation of Compound 1-3

Compound 1-3 is prepared according to the following scheme:

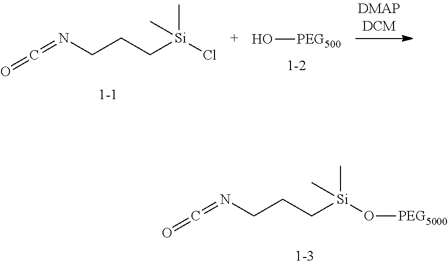

To a solution of chlorosilane 1-1 (21 mg, 120 umol) in 1 mL of anhydrous DCM, DMAP (25 mg, 205 μmol) is added. Then a solution of PEG5000-OH (1-2) inl mL of anhydrous DCM is added. Reaction is monitored by HPLC in ammonium bicarbonate buffer. After 2 h 4 mL of anhydrous ether is added, and the resulting precipitate is removed by centrifugation at room temperature. The supernatant is collected and diluted with 12 mL of anhydrous ether. The mixture is cooled in a freezer for 30 min. The precipitated product 1-3 is collected by centrifugation and removal of ether supernatant. Compound 1-3 is hydrolytically unstable; therefore the precipitate is dried under nitrogen flow and used without delay. MS m/z Calcd. For C₂₃₃H₄₆₇NO₁₁₅Si 5148. Found 142 [Si(Me)₂(CH₂)₃NCO] (M-PEG-O (5006)). ¹H-NMR (300 MHz, CDCl₃): δ 3.0-4.0 (m, 457H), 0.7-2.0 (m, 2H), 0.3-0.7 (m, 2H), −0.1-0.1 (m, 6H).

### Example 2

### Preparation of Compound 1-7

Compound 1-7 is prepared according to the following scheme:

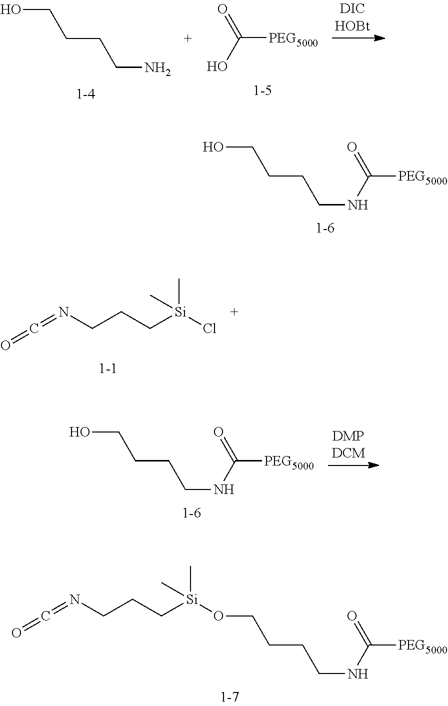

To a solution of PEG acid 1-5 (330 mg, 66 μmol) in 0.5 mL DCM, 0.5 mL of anh. DMF is added, as well as HOBt (18 mg, 132 μmol), DIEA (46 μl), and DIC (33 mg, 264 μmol). Then aminobutanol 1-4 (12 mg, 132 umol) is added. The reaction mixture is stirred for 16 h and monitored by HPLC. After completion, the mixture is purified by HPLC to give alcohol 1-6 (300 mg, 90%).

Alcohol 1-3 (72 mg, 14 μmol) is reacted with chlorosilane 1-1 (10 mg, 57 umol) according to the protocol set forth in Example 1 to give compound 1-7. MS m/z Calcd. For C₂₃₅H₄₇₀N₂O₁₁₅Si 5189. Found 288 [HOCH₂CONH(CH₂)₄OSi(Me)₂(CH₂)₃NCO] (M-PEG (4901)). ¹H-NMR (300 MHz, CDCl₃): δ 3.0-4.0 (m, 455H), 0.7-2.0 (m, 6H), 0.3-0.7 (m, 2H), −0.1-0.1 (m, 6H).

### Example 3

### Preparation of Compound 1-10

Compound 1-10 is prepared according to the following scheme:

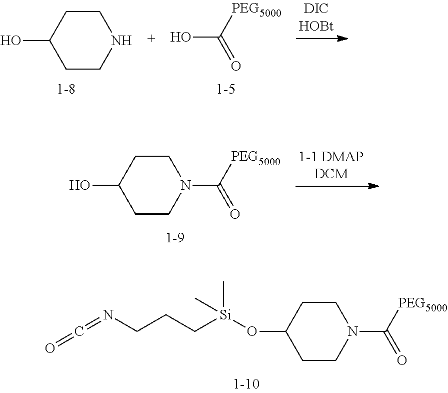

To a solution of PEG acid 1-5 (1.0 g, 200 umol) in 20 mL DCM, 2 mL of anh. DMF is added, along with HOBt (40 mg, 300 umol), DIEA (90 ul), and DIC (75 mg, 600 umol). Then a solution of alcohol 1-8 (80 mg, 800 umol) in 2 mL of DCM is added. The reaction mixture is stirred for 16 h and monitored by HPLC. After completion, the mixture is purified by HPLC to give alcohol 1-9 (647 mg, 64%).

Alcohol 1-9 (60 mg, 12 umol) is reacted with chlorosilane 1-1 (21 mg, 120 umol) according to the protocol set forth in Example 1 give compound 1-10. MS m/z Calcd. For C₂₃₆H₄₇₀N₂O₁₁₅Si 5201. Found 327 [CH₂CH₂OCH₂CON(CH₂CH₂)₂CHOSi(Me)₂(CH₂)₃NCO] (M-PEG (4874)). ¹H-NMR (300 MHz, CDCl₃): δ 3.0-4.0 (m, 456H), 0.7-2.3 (m, 6H), 0.3-0.7 (m, 2H), −0.1-0.1 (m, 6H).

### Example 4

### Preparation of Compound 1-13

Compound 1-13 is prepared according to the following scheme:

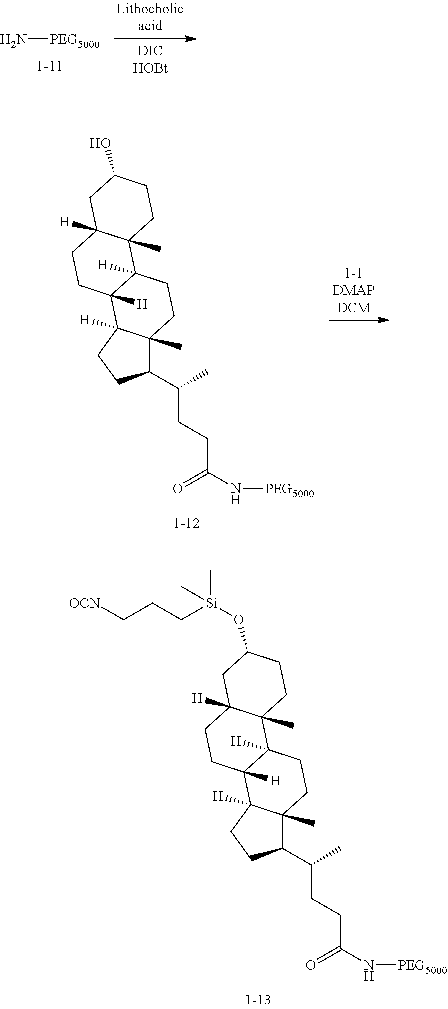

To a solution of lithocholic acid (217 mg, 576 umol) in 5 mL DCM, 1 mL of anh. DMF is added, along with HOBt (93 mg, 689 umol), DIEA (220 ul), and DIC (217 mg, 1.73 mmol). Then a solution of PEG amine hydrochloride 1-11 (2.4 g, 480 umol) and DIEA (83 ul) in 20 mL of DCM is added. The reaction mixture is stirred for 16 h and monitored by HPLC. After completion, the mixture is purified by HPLC to give alcohol 1-12 (62%).

Alcohol 1-12 (63 mg, 12 umol) was reacted with chlorosilane 1-1 (21 mg, 120 mmol) according to the protocol set forth in Example 1 to give compound 1-13. MS m/z Calcd. For C₂₅₅H₅₀₂N₂O₁₁₅Si 5461. Found 516 [NH₂-lithocholyl-OSi(Me)₂(CH₂)₃NCO] (M-PEG (4945)). ¹H-NMR (300 MHz, CDCl₃): δ 3.0-4.0 (m, 456H), 0.7-2.3 (m, 34H), 0.3-0.7 (m, 5H), −0.1-0.1 (m, 6H).

### Example 5

### Preparation of Compound 1-15

Compound 1-15 is prepared according to the following scheme:

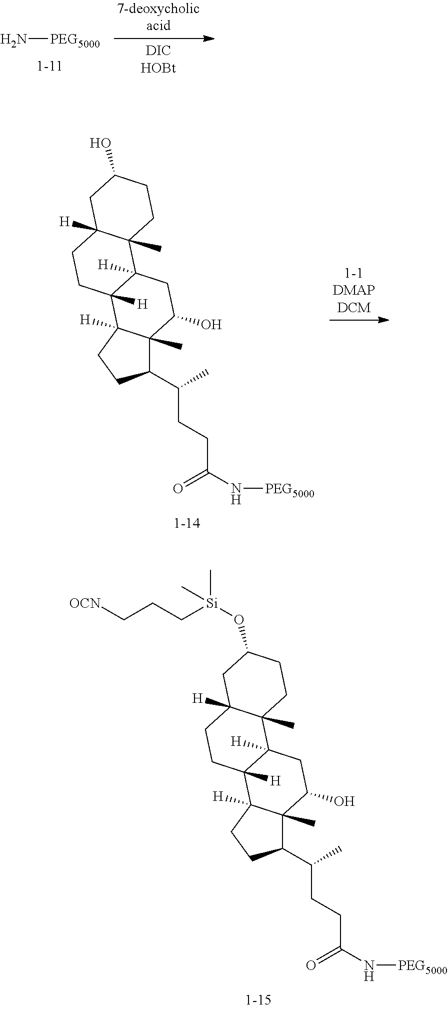

To a solution of 7-deoxycholic acid (28 mg, 72 umol) in 0.5 mL of anh. DMF, HOBt (12 mg, 89 umol), DIEA (17 ul), and DIC (40 mg, 320 umol) are added. Then a solution of PEG amine hydrochloride 1-11 (300 mg, 60 umol) and DIEA (17 ul) in 1.5 mL of DCM is added. The reaction mixture is stirred for 16 h and monitored by HPLC. After completion, the mixture is purified by HPLC to give alcohol 1-14 (60%).

Alcohol 1-14 (63 mg, 12 umol) is reacted with chlorosilane 1-1 (12.5 mg, 70 mmol) according to the protocol set forth in Example 1 to give compound 1-15. MS m/z Calcd. For C₂₅₅H₅₀₂N₂O₁₁₆Si 5477. Found 532 [NH₂-7-deoxycholyl-OSi(Me)₂(CH₂)₃NCO] (M-PEG (4945)). ¹H-NMR (300 MHz, CDCl₃): δ 3.0-4.0 (m, 458H), 0.7-2.3 (m, 32H), 0.3-0.7 (m, 5H), −0.1-0.1 (m, 6H).

### Example 6

### Preparation of Compound 1-16

Compound 1-16 is prepared according to the following scheme:

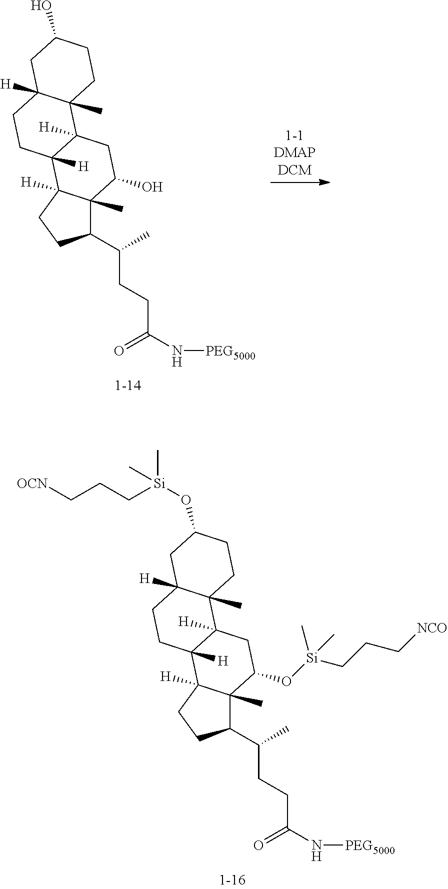

Alcohol 1-14 (63 mg, 12 umol) is reacted with chlorosilane 1-1 (25 mg, 140 mmol) according to the protocol set forth in Example 1 to give compound 1-16. MS m/z Calcd. For C₂₆₁H₅₁₃N₃O₁₁₇Si₂ 5618. Found 531 [NH₂-7-deoxycholyl-OSi(Me)₂(CH₂)₃NCO] (M-PEG (4945)-Si(Me)₂(CH₂)₃NCO (142)). ¹H-NMR (300 MHz, CDCl₃): δ 3.0-4.0 (m, 460H), 0.7-2.3 (m, 34H), 0.3-0.7 (m, 7H), −0.1-0.1 (m, 12H).

### Example 7

### Preparation of Compound 1-19

Compound 1-19 is prepared according to the following scheme:

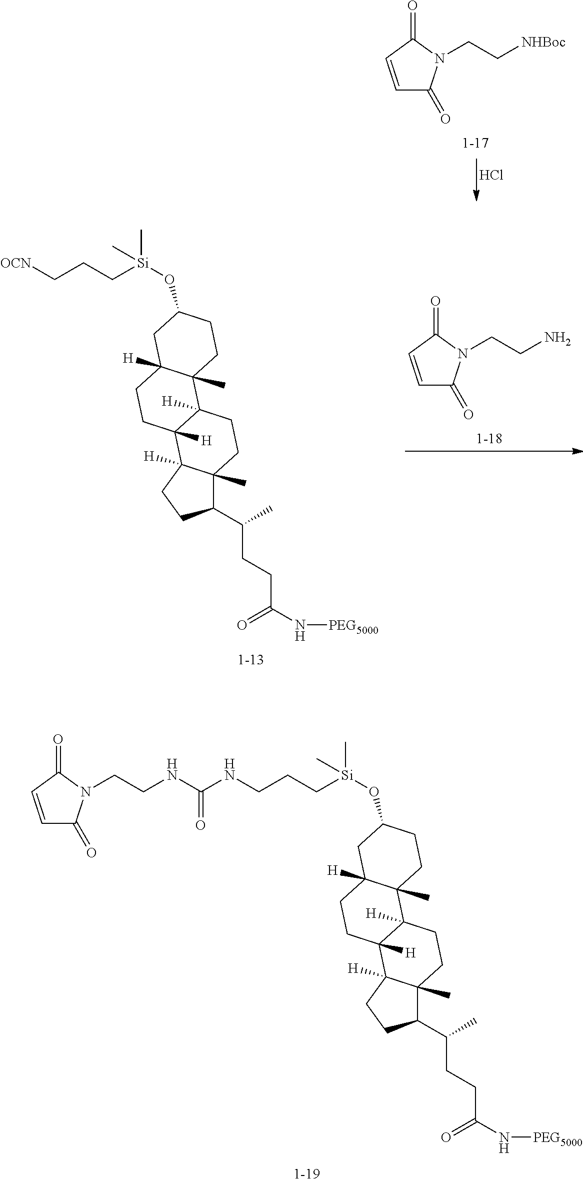

Boc protected amine 1-17 (0.6 mg, 2.5 umol) is dissolved in 30 uL of dioxane, then 890 μL of 4N HCl in dioxane is added, stirred for 2 h, then evaporated. The residue is dissolved in DCM and evaporated again to give dry amine 1-18 as hydrochloride.

Amine 1-18 is dissolved in 60 uL of anh. DMF, 2 μL of DIEA is added, then isocyanate 1-13 (2.0 umol) in 1 mL of DCM is added. The reaction is monitored by HPLC. After 3 h, the reaction mixture is diluted with 10 mL of ether, cooled in a freezer for 30 min. The precipitate is collected by centrifugation and removal of ether, then dried in a stream of nitrogen to give compound 1-19. MS m/z Calcd. For C₂₆₁H₅₁₀N₄O₁₁₇Si 5601. Found 656 [NH₂-lithocholyl-OSi(Me)₂(CH₂)₃NHCONHCH₂CH₂-maleimide] (M-PEG (4945)). ¹H-NMR (300 MHz, CDCl₃): δ 5.18 (s, 2H), 3.0-4.0 (m, 468H), 0.7-2.3 (m, 34H), 0.3-0.7 (m, 5H), −0.1-0.1 (m, 6H).

### Example 8

### Preparation of Compound 1-24

Compound 1-24 is prepared according to the following scheme:

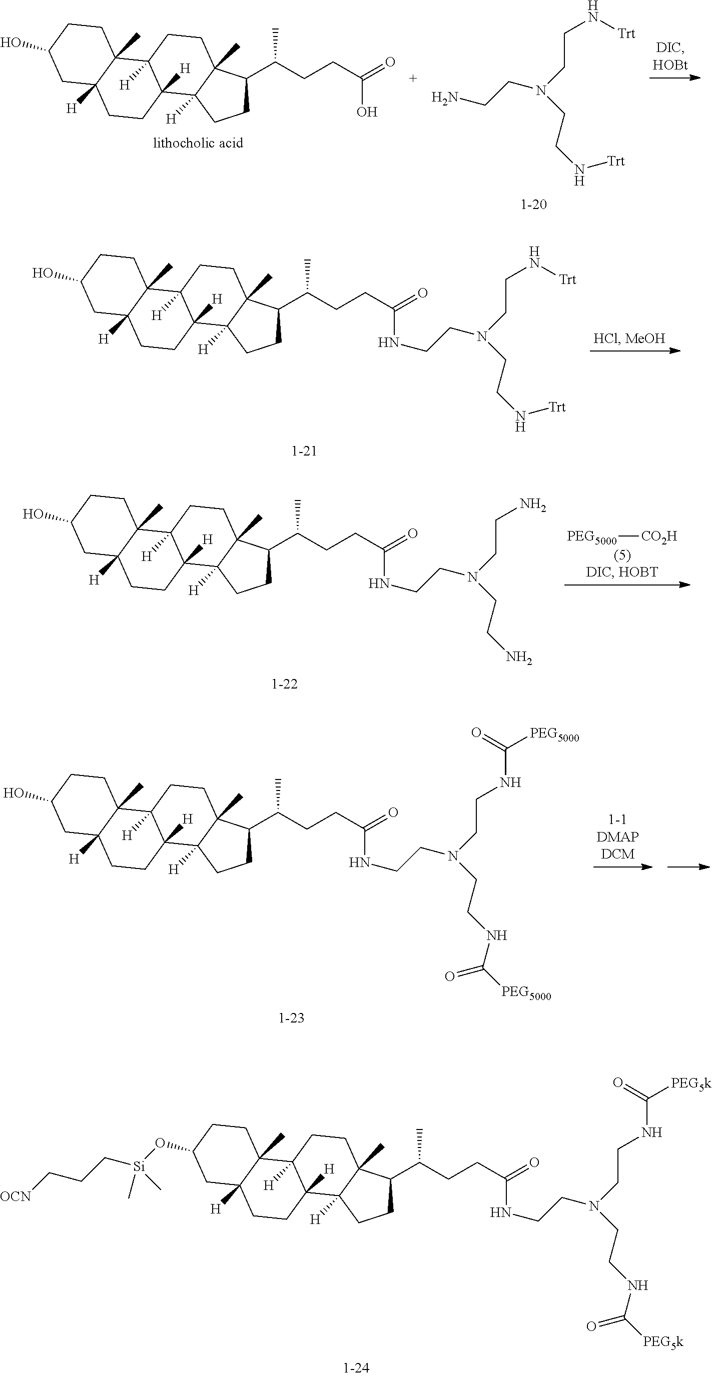

To a solution of lithocholic acid (79 mg, 210 umol) in 1.5 mL of anh. DMF, HOBt (28 mg, 210 μmol), DIEA (74 μl), and DIC (60 mg, 480 μmol) is added. Then amine 1-20 (126 mg, 200 μmol) is added. The reaction mixture is stirred for 16 h and monitored by HPLC. After completion, the mixture is diluted with water (10 mL). The precipitate of 1-21 is collected, suspended in 8 mL of MeOH, then 4 mL of conc. HCl is added and stirred for 16 h. The reaction mixture is then purified by HPLC to give amine 1-22 (60%).

To a solution of 1-22 in PEG acid 1-5 (124 mg, 25 μmol) in 1 mL of DCM, HOBt (3.5 mg, 26 μmol), DIEA (7 μl), and DIC (20 mg, 160 μmol) is added. Then a solution of amine 1-22 (7.3 mg, 12 μmol) and DIEA (7 μl) in 0.7 mL of DMF is added. The reaction mixture is stirred for 16 h and monitored by HPLC. After completion, the mixture is purified by HPLC to give alcohol 1-23 (76%).

Alcohol 1-23 (45 mg, 4.3 umol) is reacted with chlorosilane 1-1 (12.5 mg, 70 mmol) according to the protocol set forth in Example 1 to give compound 1-24. MS m/z Calcd. For C₄₈₈H₉₆₇N₅O₂₂₉Si 10591. Found 543 [CH₂CH₂NH-lithocholyl-OSi(Me)₂(CH₂)₃NCO] (M-PEG (10048)). ¹H-NMR (300 MHz, CDCl₃): δ 3.0-4.0 (m, 922H), 0.7-2.3 (m, 34H), 0.3-0.7 (m, 5H), −0.1-0.1 (m, 6H).

### Example 9

### Preparation of Compound 1-27

Compound 1-27 is prepared according to the following scheme:

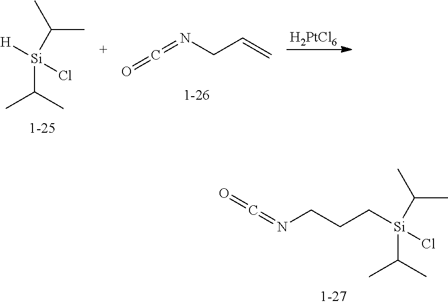

To neat chlorosilane 1-25 (2.04 g, 13.56 mmol), allylisocyanate 1-26 (1.0 mL, 11.3 mmol) is added, then H₂PtCl₆.6H₂O (30 mg) dissolved in 300 uL of DME is added. The mixture is capped and stirred at 80° C. for 16 h. HPLC shows only partial conversion. Another 4.0 g of 1-25 and 100 mg of H₂PtCl₆.6H₂O in 200 uL of DME is added, then the mixture is capped and stirred at 80° C. for 2 days. HPLC shows completed conversion. The mixture is distilled under vacuum to give the product 1-27 (452 mg, 17%) as clear oil. MS m/z Calcd. For C₁₀H₂₀ClNOSi 233. Found 206 (M−Cl+OH+H).

### Example 10

### Preparation of Compound 1-31

Compound 1-31 is prepared according to the following scheme:

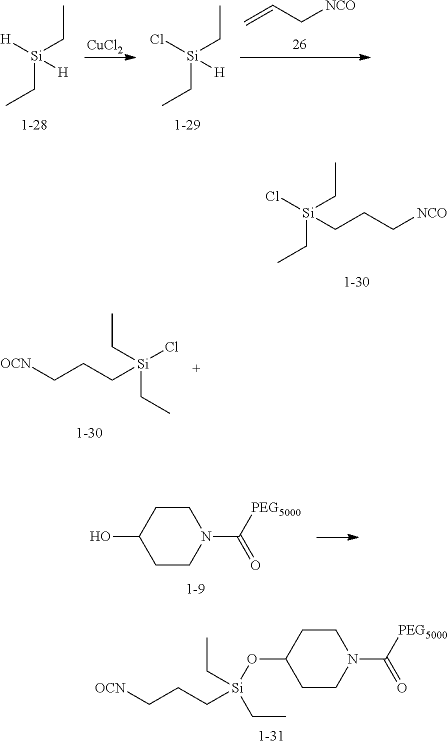

To anhydrous CuCl₂ (766 mg, 5.7 mmol), 5 mL of pentane is added, then diethylsilane 1-28 (1.0 g, 11.3 mmol) is added. The mixture is stirred with UV lamp radiation under nitrogen for 24 h to form 1-29. Then the clear solution containing >90% of 1-29 is separated from precipitated copper, and mixed with allylisocyanate 1-26 (830 mg, 10 mmol), then H₂PtCl₆.6H₂O (30 mg) dissolved in 200 uL of DME is added. The mixture is capped and stirred at 60° C. for 3 days. HPLC shows completed conversion. The mixture is distilled under vacuum to give the product 1-30 (566 mg, 24%) as clear oil.

Alcohol 1-9 (60 mg, 11.8 umol) is reacted with chlorosilane 1-30 (17 mg, 82 umol) according to the protocol set forth in Example 1 to give compound 1-31. MS m/z Calcd. For C₂₃₈H₄₇₄N₂O₁₁₅Si 5229. Found 355 [CH₂CH₂OCH₂CON(CH₂CH₂)₂CHOSi(Et)₂(CH₂)₃NCO] (M-PEG (4874)).

### Example 11

### Synthesis of PEG Maleic Anhydride 2-3

The synthesis of MPEG maleic anhydride 2-3 is carried out according to the following

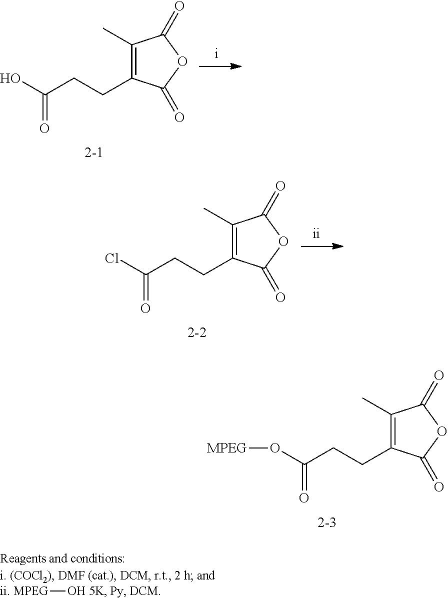

2-Propionic-3-methylmaleic anhydride 2-1 was synthesized as described in the literature (see, for example, Tetrahedron 1994, 50, 8969). Anhydride 2-1 (92 mg, 0.5 mmol) was dissolved in anhydrous DCM (10 mL). A catalytic amount of DMF (3 μL) was added and the solution was cooled in an ice bath. Oxalyl chloride (500 μL) was added dropwise via a syringe. The ice bath was removed after the addition was complete and the reaction mixture was stirred at room temperature for 2 h. The volatiles were removed under reduced pressure to give the crude acid chloride 2-2, which was used in the next step without further purification.

Monomethoxyl polyethylene glycol (MPEG; M.W. 5000, 2.5 g) was dried by azeotropic removal of toluene (2×100 mL). The dried MPEG was dissolved in anhydrous DCM (20 mL) and 1 mL of anhydrous pyridine was added. The solution of acid chloride 2-2 in DCM (5 mL) was added to this solution and the reaction mixture was stirred at room temperature for 2 h. DCM was removed under reduced pressure and the residue was purified by reverse phase HPLC (5-95% acetonitrile gradient, TFA modified mobile phase) to give compound 2-3 (1.5 g) as a white powder after lyophilization. ¹H NMR (400 MHz, CDCl₃) δ: 4.23 (t, 2H), 3.85-3.45 (m, PEG), 3.36 (s, 3H), 2.85-2.65 (m, 4H), 2.12 (s, 3H). MS m/z: Fragment Calcd. for C₁₀H₁₁O₅⁺ 211.06. Found, 211.1.

### Example 12

### Synthesis of Maleate Amides 2-5 and 2-6

The synthesis of maleate amides 2-5 and 2-6 is carried out according to the

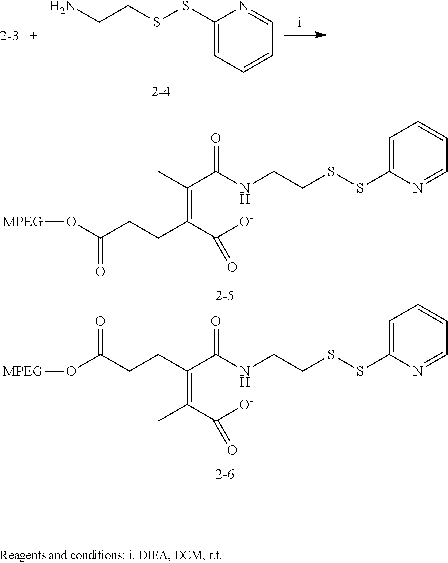

Compound 2-4 (2 mg, 11 gmol) was dissolved in anhydrous DCM (0.5 mL). DIEA (40 μL) was added. MPEG maleic anhydride 2-3 (50 mg, 10 μmol) was added to the solution and the mixture was stirred at room temperature for 20 min. The reaction mixture was cooled in an ice bath and diethyl ether (8 mL) was added under vigorous stirring. After 5 mins, the precipitated white solid was collected via filtration, washed with cold ether, and dried in vacuo for 1 h to give a mixture of compound 2-5 and 2-6 (45 mg). The solid was stored in a freezer prior to use. ¹H NMR (400 MHz, CDCl₃) δ: 8.55-8.45 (m, 1H), 7.70-7.65 (m, 2H), 7.15-7.10 (m, 1H), 4.23 (t, 2H), 3.85-3.45 (m, PEG), 3.36 (s, 3H), 3.20-2.45 (m, 8H), 1.95, 1.90 (2s, 3H). MS m/z: Fragment Calcd. for C₇H₁₀N₂S₂ 186.03. Found 186.9 (M+H⁺).

### Example 13

### Synthesis of Maleate Amides 2-8 and 2-9

The synthesis of maleate amides 2-8 and 2-9 is carried out according to the following Scheme:

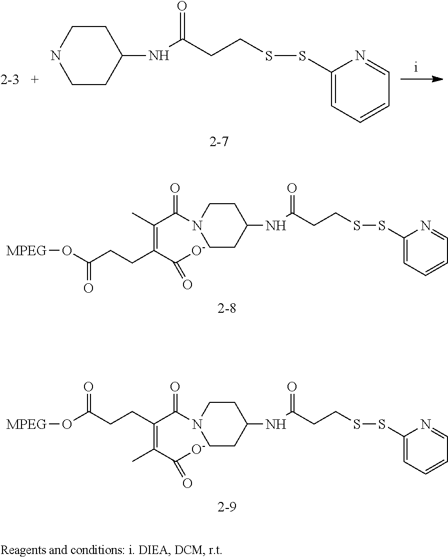

Compound 2-7 (3.3 mg, 11 μmol) was dissolved in anhydrous DCM (0.5 mL). DIEA (40 μL) was added. MPEG maleic anhydride 2-3 (50 mg, 10 μmol) was added to the solution and the mixture was stirred at room temperature for 30 min. The reaction mixture was cooled in an ice bath and diethyl ether (8 mL) was added under vigorous stirring. After 5 mins, the precipitated white solid was collected via filtration, washed with cold ether, and dried in vacuo for 1 h to give a mixture of compound 2-8 and 2-9 (42 mg). The solid was stored in a freezer prior to use. ¹H NMR (400 MHz, CDCl₃) δ: 8.50 (br s, 1H), 7.65 (br s, 2H), 7.15-7.10 (m, 1H), 4.30-4.20 (m, 2H), 3.85-3.45 (m, PEG), 3.36 (s, 3H), 3.10-2.45 (m), 2.15, 1.95 (2s, 3H). MS m/z: Fragment Calcd. for C₁₃H₁₉N₃OS₂ 297.1. Found 298.0 (M+H⁺).

### Example 14

### Preparation of Compounds 2-10-2-21

The synthesis of compounds 2-10 through 2-21 is carried out according to the following Scheme:

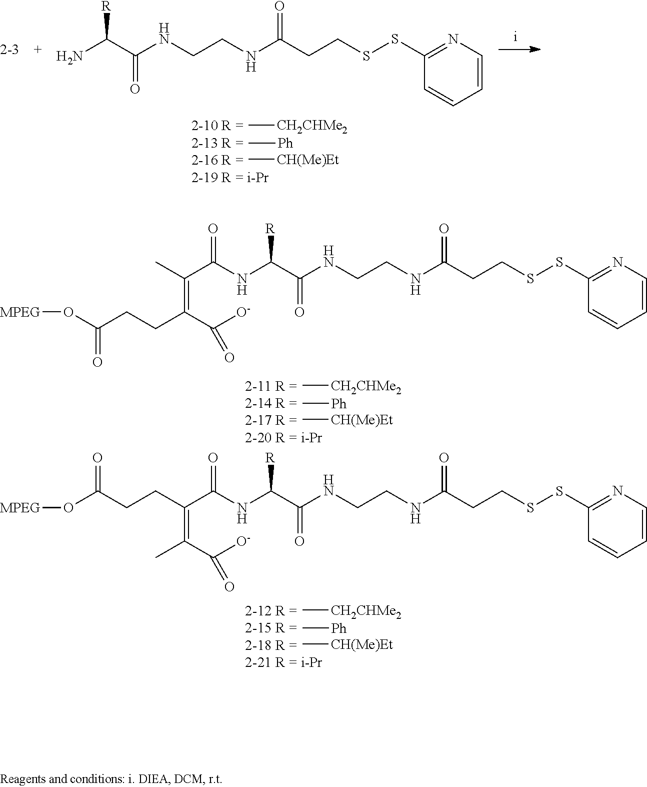

Compound 2-10 (4.1 mg, 11 μmol) was dissolved in anhydrous DCM (0.5 mL). DIEA (40 μL) was added. MPEG maleic anhydride 2-3 (50 mg, 10 μmol) was added to the solution and the mixture was stirred at room temperature for 16 h. The reaction mixture was cooled in an ice bath and diethyl ether (8 mL) was added under vigorous stirring. After 5 mins, the precipitated white solid was collected via filtration, washed with cold ether, and dried in vacuo for 1 h to give a mixture of compound 2-11 and 2-12 (46 mg). The solid was stored in a freezer prior to use. ¹H NMR (400 MHz, CDCl₃) δ: 8.45 (dd, 1H), 8.20 (br s, 1H), 7.70-7.55 (m, 2H), 7.10-6.95 (m, 1H), 6.20 (br s, 1H), 4.40-4.35 (m, 1H), 4.30-4.20 (m, 2H), 3.85-3.45 (m), 3.36 (s, 3H), 3.00 (t, 2H), 1.95, 1.90 (2s, 3H), 0.90 (dd, 6H). MS m/z: Fragment Calcd. for C₁₆H₂₆N₄O₂S₂ 370.15. Found 371.3 (M+H⁺).

A mixture of compound 2-14 and 2-15 was synthesized by the same procedure as described for compound 2-11 and 2-12. ¹H NMR (400 MHz, CDCl₃) δ: 8.45 (dd, 1H), 8.20 (br s, 1H), 7.70-7.55 (m, 2H), 7.40-7.20 (m, 5H), 7.10-6.95 (m, 1H), 6.60 (br s, 1H), 5.35 (d, 1H), 4.25-4.20 (m, 2H), 3.85-3.10 (m), 1.95, 1.90 (2s, 3H). MS m/z: Fragment Calcd. for C₁₈H₂₂N₄O₂S₂ 390.12. Found 391.0 (M+H⁺).

A mixture of compound 2-17 and 2-18 was synthesized by the same procedure as described for compound 2-11 and 2-12. ¹H NMR (400 MHz, CDCl₃) δ: 8.45 (dd, 1H), 8.20 (br s, 1H), 7.70-7.55 (m, 2H), 7.10-6.95 (m, 1H), 6.20 (br s, 1H), 4.40-4.35 (m, 1H), 4.30-4.20 (m, 2H), 3.85-3.45 (m), 3.36 (s, 3H), 3.00 (t, 2H), 1.95, 1.90 (2s, 3H), 0.90-0.70 (m, 6H). MS m/z: Fragment Calcd. for C₁₆H₂₆N₄O₂S₂ 370.15. Found 371.1 (M+H⁺).

A mixture of compound 2-20 and 2-21 was synthesized by the same procedure as described for compound 2-11 and 2-12. ¹H NMR (400 MHz, CDCl₃) δ: 8.45 (dd, 1H), 8.20 (br s, 1H), 7.70-7.55 (m, 2H), 7.10-6.95 (m, 1H), 4.40-4.35 (m, 1H), 4.30-4.20 (m, 2H), 3.85-3.45 (m), 3.36 (s, 3H), 3.00 (t, 2H), 1.95, 1.90 (2s, 3H), 0.90-0.70 (m, 6H). MS m/z: Fragment Calcd. for C₁₅H₂₄N₄O₂S₂ 356.13. Found 357.0 (M+H⁺).

### Example 15

### Preparation of Compound 3-7 and 3-8

The synthesis of compounds 3-7 and 3-8 is carried out according to the following Schemes:

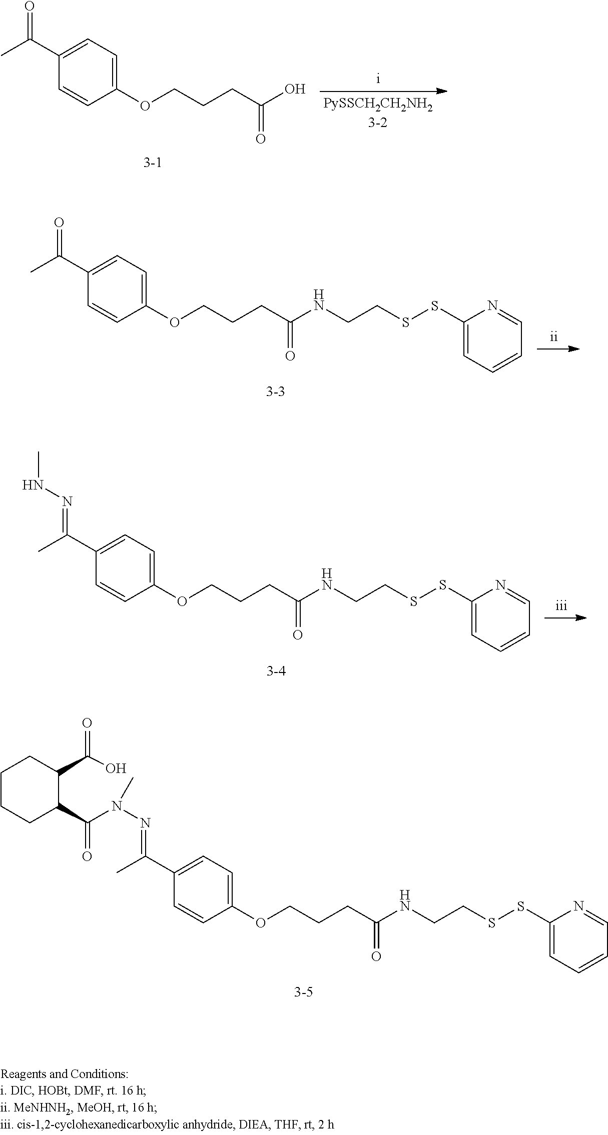

The carboxylic acid 3-1 was synthesized according to a literature procedure (Bioconjugate Chem., 2002, 13, 47-58). To a mixture of compound 3-1 (206 mg, 1 mmol), amine 3-2 (186 mg, 1 mmol), HOBt (154 mg, 1 mmol) in DMF (5 mL) was added DIC (1.5 mmol) and the reaction mixture was stirred at room temperature for 4 h. The solvents were removed under reduced pressure, and the crude product was dissolved in DCM (50 mL). The DCM solution was washed successively with saturated aqueous sodium bicarbonate (30 mL) and brine (20 mL). The organic phase was concentrated and the residue was purified by reverse phase HPLC to give compound 3-3 as a white powder after lyophilization (304 mg, 78%). MS m/z: Calcd. for C₁₀H₂₂N₂O₃S₂ 390.1. Found, 391.4 (M+H⁺).

A 40 mg sample of compound 3-3 was dissolved in anhydrous MeOH (1 mL). 10 eq of methylhydrazine (10 mmol) was added and the reaction was stirred at room temperature for 16 h. The reaction mixture was evaporated to dryness and the residue was dissolved in THF (2 mL). Then, 100 μL of DIEA was added followed by addition of cis-1,2-cyclohexanedicarboxylic anhydride (75 mg) and the reaction was allowed to stir at room temperature for 4 h. The solvents were removed under reduced pressure and the crude product was purified by reverse phase HPLC to yield compound 3-5 as a white power after lyophilization (18 mg). MS m/z: Calcd. for C₂₈H₃₆N₄O₅S₂ 572.2. Found 573.3 (M+H⁺).

Compounds 3-7 and 3-8 are then prepared according to the following Scheme:

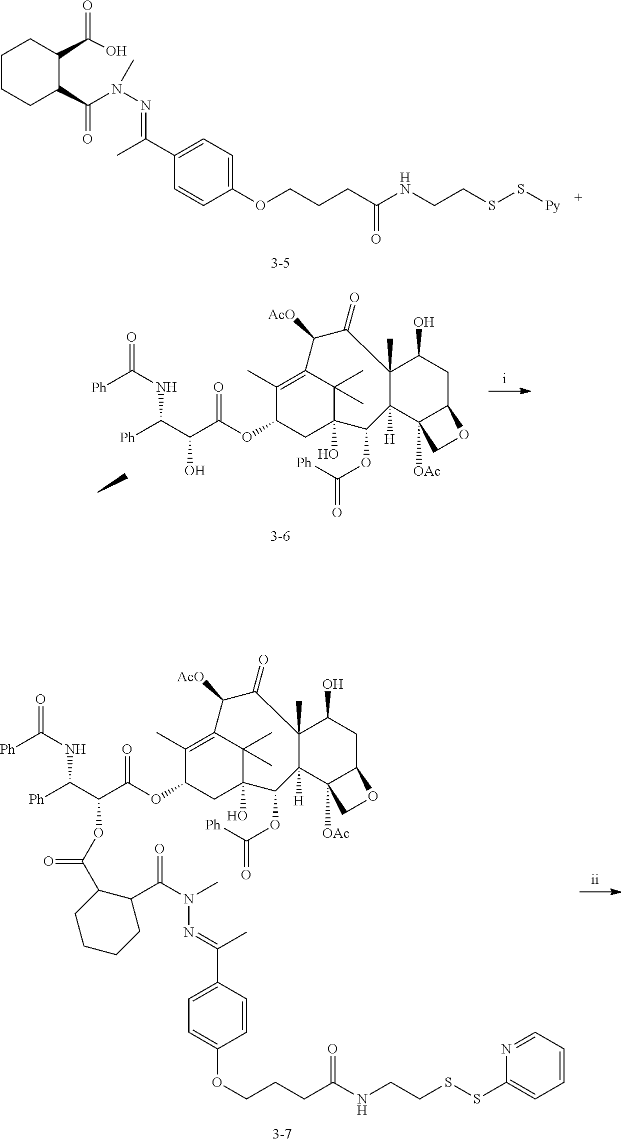

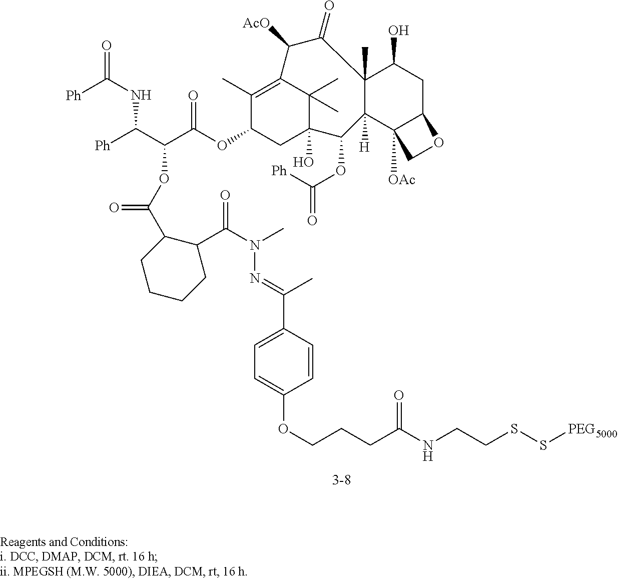

A solution of compound 3-5 (10 mg) and paclitaxel 3-6 (9 mg) in anhydrous DCM (1 mL) was stirred at room temperature for 16 h with DMAP (2 mg) and DCC (5 mg). The precipitates were filtered off and the filtrate was concentrated to dryness. The crude product was purified by reverse phase HPLC to give compound 3-7 as a white powder after lyophilization (6 mg). MS m/z: Calcd. for C₇₅H₈₅N₅O₁₈S₂ 1407.5. Found 1408.8 (M+H⁺).

A sample of compound 3-7 (3 mg) was dissolved in DCM (1 mL) and 12 mg of methoxy polyethylene glycol thiol (M.W. 5000) was added. DIEA (2 μL) was added to the reaction and the mixture was allowed to stir at room temperature. The reaction was monitored by HPLC. After 16 h, most of compound 3-7 and MPEG-SH was consumed. The solvents were removed under reduced pressure and the crude product was purified by reverse phase HPLC to give the pegylated compound 3-8 as a white powder (6 mg).

### Example 16

### Preparation of Compound 3-13 and 3-14

The synthesis of compounds 3-13 and 3-14 is carried out according to the following Schemes:

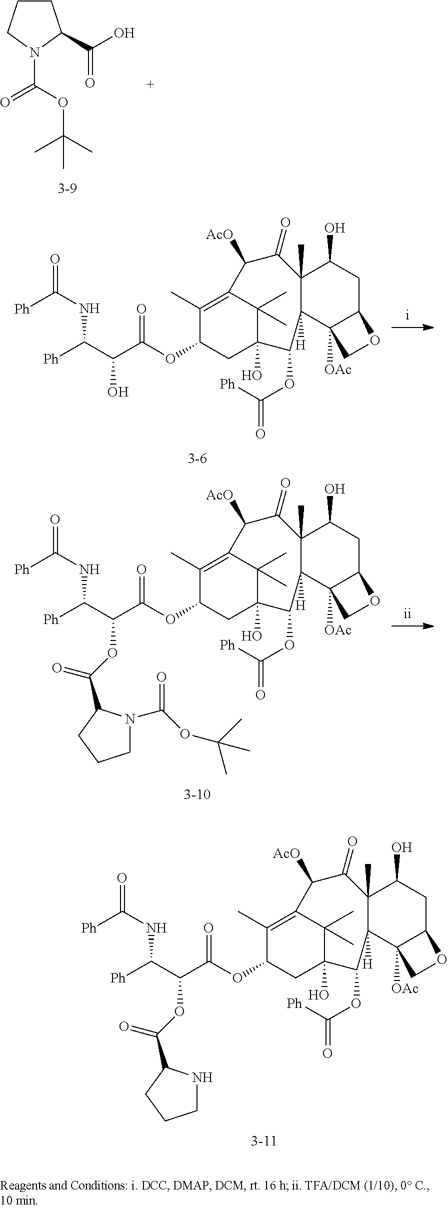

To a stirred solution of Boc-Proline (3-9, 5 mg), paclitaxel (3-6, 17 mg) and DMAP (3 mg) in anhydrous DCM (2 mL) was added DCC (8 mg) and the reaction mixture was stirred at room temperature for 16 h. The precipitated urea was filtered off. The filtrate was diluted with 40 mL of DCM and the DCM solution was washed with 1N aqueous HCl (20 mL) and brine (20 mL). The organic phase was dried and evaporated to dryness to give the crude product 3-10 which was used directly for the next step without further purification. MS m/z: Calcd. for C₅₇H₆₆N₂O₁₇ 1050.4. Found 1051.4 (M+H⁺).

The crude compound 3-10 was dissolved in DCM (2 mL) and the solution was cooled in an ice bath. TFA (0.2 mL) was added and the reaction mixture was stirred at 0° C. for 10 min. Toluene (5 mL) was added and the mixture was evaporated under reduced pressure. The residue was purified by reverse phase HPLC to give compound 3-11 (9 mg) as a white powder after lyophilization. MS m/z: Calcd. for C₅₂H₅₈N₂O₁₅ 950.4. Found, 951.5 (M+H⁺).

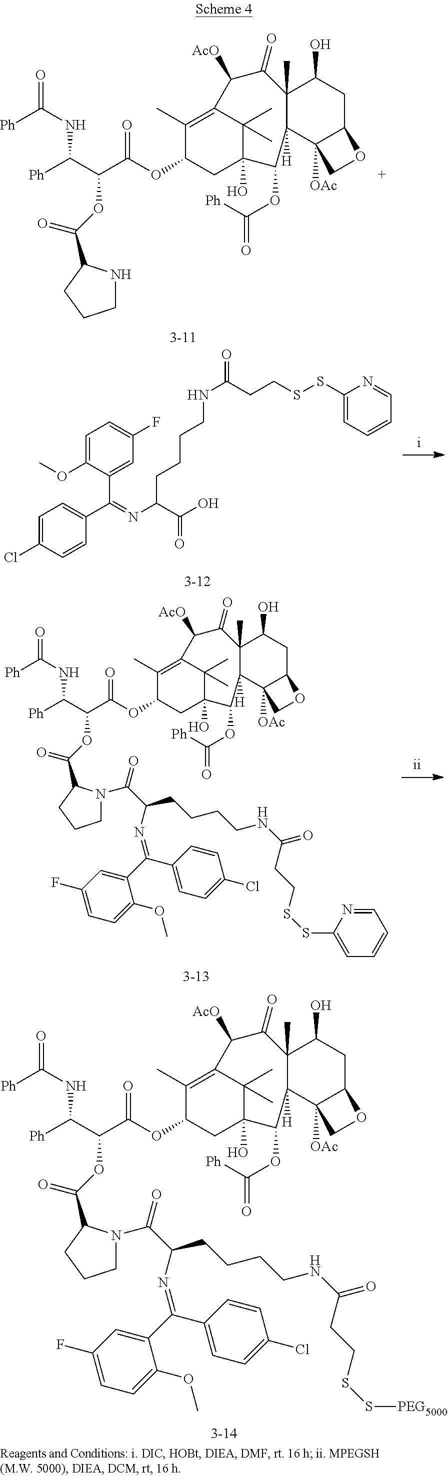

A sample of compound 3-11 (8 mg) and compound 3-12 (6 mg) was dissolved in anhydrous DMF (1 mL) in the presence of DIEA (15 μL). HOBt (2 mg) and DIC (12 μL) were added and the reaction mixture was allowed to stir at room temperature for 16 h. LC/MS indicated the completion of the reaction and the crude mixture was purified directly by reverse phase HPLC. The desired product 3-13 was obt3-7 and 3-8ained as a white solid (6 mg) after lyophilization. MS m/z: Calcd. for C₈₀H₈₅ClFN₅O₁₈S₂ 1521.5. Found 1522.8 (M+H⁺).

A sample of compound 3-14 (2 mg) was dissolved in DCM (1 mL) and 10 mg of methoxy polyethylene glycol thiol (M.W. 5000) was added. DIEA (2 μL) was added to the reaction and the mixture was allowed to stir at room temperature. The reaction was monitored by HPLC. After 16 h, most of compound 3-13 was consumed. The solvents were removed under reduced pressure and the crude product was purified by reverse phase HPLC to give the pegylated compound 3-14 as a white powder (5 mg).

### Example 17

### TCPC Particle Size, Pegylation, and Stability

### TCPC/siRNA Particle Formulation

Lyophilized TCPCs are reconstituted to a final concentration of 1 mM using 1 mM EDTA to create master stocks, which are then aliquoted and stored at −20° C. From the master stock, 10×TCPC working stocks are created by diluting appropriate amounts of the master in H₂O. For a standard 50 μL formulation, 5 μL of 200 μM TCPC (10×) is added to 45 μL of 1.11 μM siRNA (1.11x) in H₂O, creating a final 20:1 molar ratio of TCPC:siRNA (20 μM:1 μM). Particle formation in varying buffers (e.g. HEPES, PBS, etc.) can be achieved by replacing 5 μL of H₂O in the siRNA solution with 5 μL of 10× concentration buffer. The resulting particles are allowed to equilibrate for 15 min at room temperature.

TCPC Pre-PEGylation with Maleimide-PEG

Maleimide-PEG stocks (1 mM) are prepared in H₂O and stored at 4° C. PEGylation percentages are based on moles of PEG/moles of TCPC. For example, to prepare 20 μL of a 10% pre-mPEGylated TCPC stock (200 μM in this example), 4 μL of 1 mM TCPC and 4 μL of 100 μM mPEG are added to 12 μL of H₂O. The solution is incubated for 15 min at room temperature to allow PEGylation. The pre-PEGylated stocks are then used as the 10×TCPC stocks in the particle formulation protocol.

TCPC Pre-PEGylation with Acid-Labile PEGs

Acid-labile PEGs 2-5, 6, 2-8, 9, 2-11, 12, 2-14, 15, 2-17, 18 are stored at −20° C. in 10 mM HEPES. PEGs 1-3, 1-7, 1-10, 1-13, 1-15, 1-16, 1-19, 1-24, 1-31 are stored at −80° C. in anhydrous DMSO. To prepare 20 μL of a 100% acid-labile pre-PEGylated TCPC stock (200 μM in this example), 4 μL of 1 mM TCPC is added to 10 μL of H₂O, followed by 2 μL of 100 mM HEPES pH 7.2 (10×), and 4 μL of 1 mM PEG (in 10 mM HEPES or DMSO). The solution is incubated for 15 min at room temperature to allow PEGylation.

For particle formation requiring final TCPC concentrations between about 20-100 μM, a 2× protocol was employed. In this protocol, both TCPC pre-PEGylation and siRNA solutions were prepared at 2× concentrations in 10 mM HEPES pH 7.2. Particles were formed by mixing equal volumes of 2×TCPC and 2× siRNA and equilibrating for 15 min at room temperature.

### Particle Size Measurements

Particle size was measured by dynamic light scattering using a DelsaNano C instrument (Beckman-Coulter). Each sizing run subjects the sample to 50 measurements over 3 minutes. The data are fit using the Cumulants method to produce an average particle diameter and polydispersity.

When TCPC is complexed with siRNA in H₂O or a neutral buffer such as HEPES, small (≦150 nm), stable particles are formed. However, these small particles will aggregate when exposed to physiological concentrations of salt. This aggregation can be prevented by incorporating polyethylene glycol (PEG). Numerous PEG constructs described herein have unique chemistries enabling reversible or irreversible PEGylation of TCPCs. Irreversible PEGylation is exemplified by maleimide-PEG (mPEG), which reacts with TCPC thiols to form stable PEGylated particles. However, high degrees of stable PEGylation can be inhibitory to TCPC:siRNA function. Therefore, it is desirable to engineer reversible PEGylating agents. According to one aspect of the present invention, reversible PEG constructs have been developed that take advantage of the acidic environment of the endosomal compartment to release PEG from the TCPC/siRNA particles.

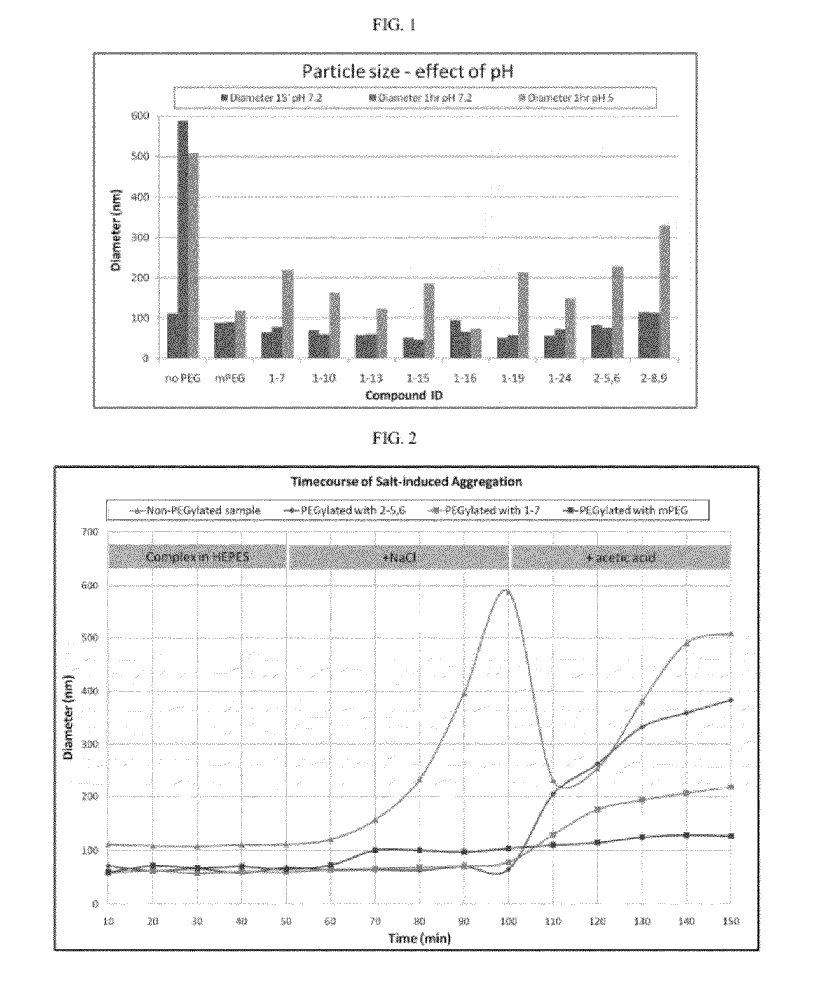

PEGylation of TCPC prior to complexing with siRNA confers salt stability to the resulting TCPC/siRNA particles. FIG. 1 shows the stability for particle samples formed with non-PEGylated TCPC or TCPC pre-PEGylated with reversible or irreversible PEG constructs. Non-PEGylated particles aggregate quickly in salt-containing buffer (150 mM NaCl). In contrast, particles are resistant to salt induced aggregation when PEGylated with either mPEG or the reversible acid-labile PEGs and particle size remains stable (FIG. 1, 15 min and 1 hr timepoints). However, when the pH of the particle solution is lowered to ˜5, the acid-labile PEGs are shed from the particles, resulting in aggregation.

FIG. 2 illustrates PEG hydrolysis and resulting particle aggregation in real time. Non-PEGylated particles (green line) are stable in HEPES buffer, but aggregate quickly upon addition of 150 mM NaCl. In contrast, particles are resistant to salt induced aggregation when PEGylated with either mPEG (blue line) or acid-labile PEGs 2-5, 6 and 1-7 (red line and orange line, respectively; see FIG. 2, timepoints 50-100). When the pH of the particle solution is lowered to ˜5, PEG is shed from the particles containing acid-labile PEGs 2-5, 6 and 1-7, resulting in aggregation. This specific reversible PEGylation is very useful to maintain the function of particles taken into the cell via endocytosis.

### Example 18

### Hydrolysis Rates of Exemplary Compounds

The ability to hydrolytically release PEG from constructs according to the invention, as a function of the pH to which said constructs are subjected was tested. Results are summarized in the following table:

| | | Hy- | Hy- |
|---|---|---|---|
| | | drol- | drol- |
| | | y- | y- |
| | | sis | sis |
| | | T_{1/2} | T_{1/2} |
| Com- | | (@ | (@ |
| pound | | pH | pH |
| ID | Structure | 7.4) | 5) |
| 1-3 | 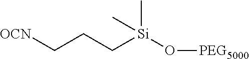 | 8 min | <5 min |
| 1-7 | 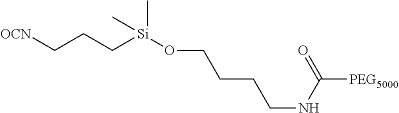 | 22 min | <5 min |
| 1-10 | 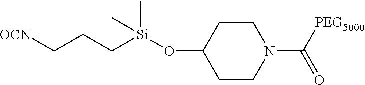 | 3.5 h | <5 min |
| 1-13 | 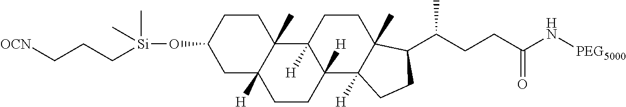 | 17 h | <5 min |
| 1-15 | 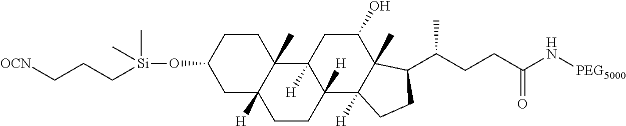 | 1.3 d | <5 min |
| 1-16 | 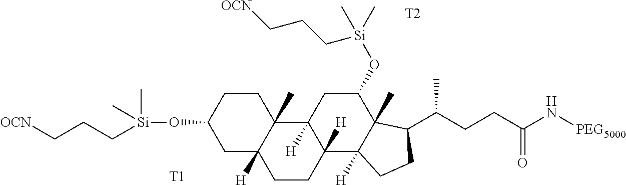 | T1 = 1.5 d T2 >10d | T1 <5 min T2 = 13 h |
| 1-19 | 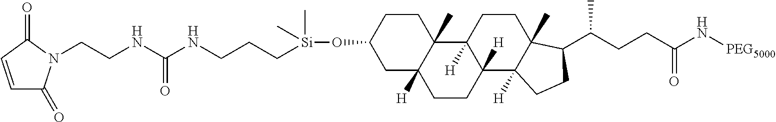 | 1.5 h | <5 min |
| 1-24 | 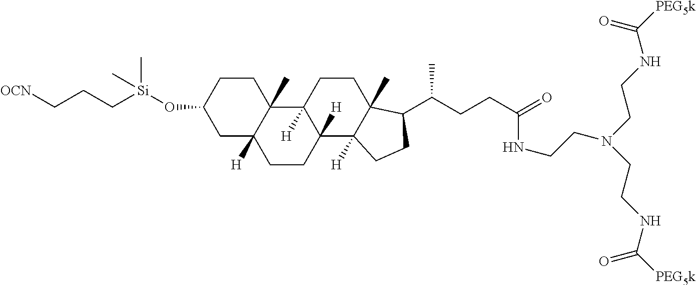 | 5 h | 1 min |
| 1-31 | 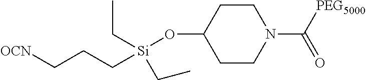 | 5 d | 13 h |
| 2-5, 6 | 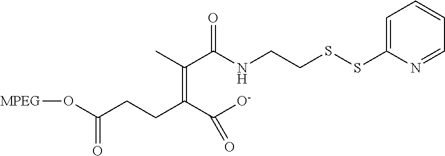 | 16 h | 5 min |
| 2-8, 9 | 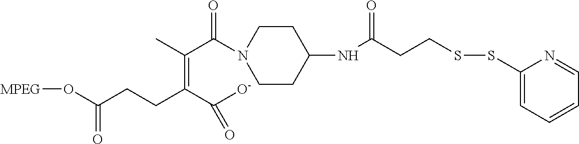 | 16 h | <5 min |
| 2-11, 12 | 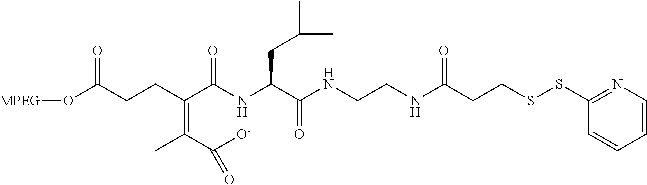 | 24 h | 1 h |
| 2-14, 15 | 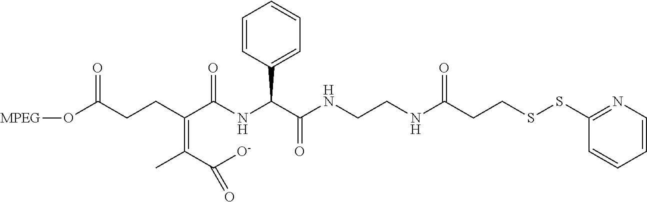 | 24 h | 30 min |
| 2-17, 18 | 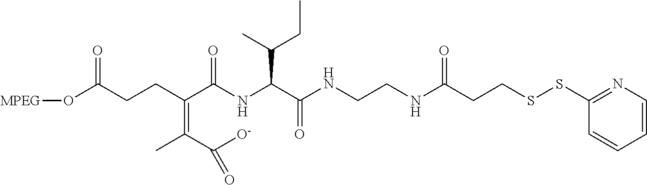 | ND | ND |
| 2-20, 21 | 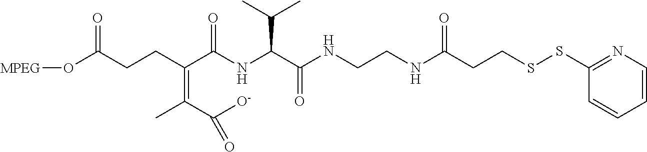 | ND | ND |

Review of the preceding table indicates that a number of constructs according to the present invention display substantially different stabilities at pH 5 as opposed to pH 7.4, thereby facilitating release of one component from invention constructs by merely subjecting the construct to a different pH environment.

### Example 19

### Knockdown Experiments

HEK293 cells (American Type Culture Collection) were cultured in Dulbecco's Modified Eagle's Medium supplemented with 10% fetal bovine serum, 10 mM HEPES and 1× Penicillin/Streptomycin at 37° C./5% CO₂. For knockdown experiments, a stable clonal cell line was generated that expresses synthetic firefly luciferase 2 (Photinus pyralis) from the pGL4 Luciferase Reporter Vector (Promega). The reporter cell line was maintained in Dulbecco's Modified Eagle's Medium supplemented with 10% fetal bovine serum, 10 mM HEPES, 1× Penicillin/Streptomycin and 400 μg/ml G418 at 37° C./5% CO₂.

Cells were seeded in 24-well plates at 25,000 cells per well or in 96-well plates at 5,000 cells per well 16-24 h prior to transfection. Particles were formed and added to cells with a final siRNA concentration of 100 nM and varying PEGylated TCPC concentrations ranging from 250 nM to 2 μM on cells (2.5:1-20:1 molar ratio) for 4 hrs at 37° C./5% CO₂ in serum-free medium after which 2 volumes of growth medium were added. Cells were placed back in the incubator and after 68-72 hrs were processed for a Luciferase Assay or qRT-PCR. For controls, Lipoplexes were prepared with the same final concentration of siRNA using Lipofectamine 2000 (Invitrogen) according to the manufacturer's instructions.

Luciferase activity in relative luminescence units (RLUs) was measured using a Molecular Devices SpectraMax M5 Microplate Reader and the Steady-Glo© Luciferase Assay System (Promega) according to manufacturer's instructions.

For qRT-PCR, total RNA was extracted using Omega Bio-Tek E.Z.N.A. MicroElute Total RNA Kit for Small Samples of Cells and Tissues. cDNA was generated using gScript™ cDNA SuperMix (Quanta Biosciences). qRT-PCR was performed on the Applied Biosystems StepOne™ system using TaqMan chemistry in a Comparative C_{T} Experiment. Reduction of luciferase 2 mRNA was determined by normalizing to a beta-actin control using the TaqMan® Gene Expression Master Mix (Applied Biosystems) using Applied Biosystems StepOne™ software.

### Oligos Employed:

| Target | Oligo Name | Sequence |
|---|---|---|
| Luciferase 2 (sense) | RGO 52TT | 5′ CCUACGCCGAGUACUUCGATT 3′ |
| | | (SEQ ID NO: 1) |
| Luciferase 2 (antisense) | RGO 53TT | 5′ UCGAAGUACUCGGCGUAGGTT 3′ |
| | | (SEQ ID NO: 2) |
| Luciferase 2 (scrambled 1) | RGO 54TT | 5′ GGCACUCAUCGACUCGUACTT3′ |
| | | (SEQ ID NO: 3) |
| Luciferase 2 (scrambled 2) | RGO 55TT | 5′ GUACGAGUCGAUGAGUGCCTT 3′ |
| | | (SEQ ID NO: 4) |

q-PCR Assays Employed:

| Target | Primer/Probe | |
|---|---|---|
| Luciferase 2 | Forward Primer | AAGGGCTGCAAAAGATCC (SEQ ID NO: 5) |
| | Reverse Primer | GTGGCAAATGGGAAGTCA (SEQ ID NO: 6) |
| | Probe | /56-FAM/ATAGCAAGACCGACTACCAGGGC/3IABLFQ/ |
| | | (SEQ ID NO: 7) |
| Beta-actin | Forward Primer | TTGGCAATGAGCGGTTC (SEQ ID NO: 8) |
| | Reverse Primer | GTTGGCGTACAGGTCTTT (SEQ ID NO: 9) |
| | Probe | /56-FAM/TTCCTGGGCATGGAGTCCTGT/3IABLFQ/ |
| | | (SEQ ID NO: 10) |

Knockdown Capabilities of TCPCs Modified with PEG Containing the Following Linkers:

| Compound ID | Knockdown with 70-100nm particles |
|---|---|
| mPEG | + |
| 1-3 | ND |
| 1-7 | +++ |
| 1-10 | +++ |
| 1-13 | +++ |
| 1-15 | +++ |
| 1-16 | +++ |
| 1-19 | +++ |
| 1-24 | +++ |
| 1-31 | ND |
| 2-5,6 | +++ |
| 2-8,9 | n.d. |
| 2-11, 12 | ND |
| 2-14, 15 | ND |
| 2-17, 18 | ND |
| 2-20, 21 | ND |

ND = not determined
+ = 0%/15% knockdown
++ = 15-30% knockdown
+++ = >30% knockdown

TCPC modified with PEG via an irreversible linker (i.e., a linker that is not labile at acidic pH) shows essentially no knock down activity. Irreversible PEGylation is exemplified by maleimide-PEG (mPEG), which reacts with TCPC thiols to form stable PEGylated particles. TCPCs linked to PEG via an acid labile linker are active as delivery agents of siRNA.

Although the invention has been described with reference to embodiments and examples, it should be understood that various modifications can be made without departing from the spirit of the invention.

All references cited herein are hereby expressly incorporated by reference in their entireties. Where reference is made to a uniform resource locator (URL) or other such identifier or address, it is understood that such identifiers can change and particular information on the internet can be added, removed, or supplemented, but equivalent information can be found by searching the internet. Reference thereto evidences the availability and public dissemination of such information.

That which is claimed is:

1. A multifunctional linker having the structure: 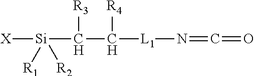
wherein:
X is a leaving group selected from the group consisting of —Cl, —Br, —I and —OSO₂R, wherein R is an optionally substituted lower alkyl, an optionally substituted aryl, or an optionally substituted heteroaryl;
R₁ and R₂ are independently optionally substituted lower alkyl, optionally substituted aryl, or optionally substituted heteroaryl;
R₃ and R₄ are independently hydrogen or optionally substituted lower alkyl, or, R₃ and R₄, taken together, are C₁-C₅ alkylene or substituted alkylene; and
L₁ is a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof, or R₃ and L₁, or R₄ and L₁, taken together, are C₁-C₅ alkylene or substituted alkylene;
provided, however, when R₃ and R₄ are hydrogen, L₁ is a covalent bond, methylene, ethylene, —CH₂C(═O)—, or —(CH₂)₈C(═O)—, and X is chloro, at least one of R₁ and R₂ is not methyl.

2. The linker of claim 1 wherein L₁ is a covalent bond, C₁-C₆ alkylene, substituted C₁-C₆ alkylene, C₂-C₆ alkenylene, substituted C₂-C₆ alkenylene, arylene, substituted arylene, heteroarylene, substituted heteroarylene, C₃-C₇ cyloalkylene, or substituted C₃-C₇ cycloalkylene, or a combination of any two or more thereof.

3. The linker of claim 1 wherein R₁ is methyl, ethyl, n-propyl, isopropyl, n-butyl, isobutyl or t-butyl, R₂ is methyl, ethyl, n-propyl, isopropyl, n-butyl, isobutyl or t-butyl, R₃ is hydrogen, methyl or ethyl, and R₄ is hydrogen, methyl or ethyl.

4. The linker of claim 1 wherein R₁ is methyl, ethyl, n-propyl, isopropyl, n-butyl, isobutyl or t-butyl, R₂ is methyl, ethyl, n-propyl, isopropyl, n-butyl, isobutyl or t-butyl, and R₃ and R₄ cooperate to form a C₃-C₇ cycloalkylene or substituted C₃-C₇ cycloalkylene ring.

5. The linker of claim 1 wherein R₁ is methyl, ethyl, n-propyl, isopropyl, n-butyl, isobutyl or t-butyl, R₂ is methyl, ethyl, n-propyl, isopropyl, n-butyl, isobutyl or t-butyl, and R₃ and L₁ cooperate to form a C₄-C₇ cycloalkylene or substituted C₄-C₇ cycloalkylene ring.

6. The linker of claim 1 wherein R₁ is methyl, ethyl, n-propyl, isopropyl, n-butyl, isobutyl or t-butyl, R₂ is methyl, ethyl, n-propyl, isopropyl, n-butyl, isobutyl or t-butyl, and R₄ and L₁ cooperate to form a C₃-C₇ cycloalkylene or substituted C₃-C₇ cycloalkylene ring.

7. A construct obtained by displacing X of the linker of claim 1 with alcohol A-OH or carboxylic acid A-CO₂H, thereby producing the structure: 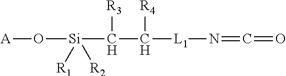
wherein:
A is an organic moiety containing at least 5 up to about 100 carbon atoms, excluding alcohols A-OH selected from the group consisting of benzyl alcohol, 2-methylphenol, 3-methylphenol, and 1-octadecanol.

8. The construct of claim 7 wherein alcohol A-OH or carboxylic acid A-CO₂H alone has known biological activity or therapeutic activity.

9. The construct of claim 7 wherein alcohol A-OH or carboxylic acid A-CO₂H is selected from the group consisting of polyethylene glycol, a polyethylene glycol derivative, a sterol, a sterol derivative, cholic acid, a cholic acid derivative, a secosteroid, a monoglyceride, a diglyceride, a phospholipid, a phospholipid derivative, a fatty acid, a fatty acid derivative, sirolimus, everolimus, paclitaxel, docetaxel, camptothecin, epothilone, an epothilone derivative, doxorubicin, a maytansinoid, an ansamycin, a retinoid, a tocopherol, a tocotrienol, biotin, a vitamin B compound, duocarmycin, a duocarmycin derivative, auristatin, and an auristatin derivative.

10. A construct having the structure:
(a) A-L₂-B—Y—C-L₃-Z
wherein:
A is a biologically compatible material containing at least 4 carbon atoms;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from: 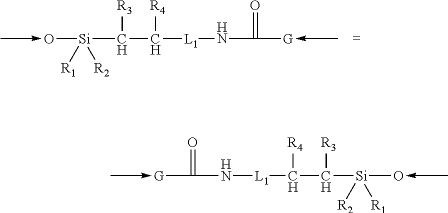
wherein:
R₁ and R₂ are independently optionally substituted lower alkyl, optionally substituted aryl, or optionally substituted heteroaryl;
R₃ and R₄ are independently hydrogen or optionally substituted lower alkyl, or, R₃ and R₄, taken together, are C₁-C₅ alkylene or substituted alkylene;
L₁ is a covalent bond, or a linker selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof, or R₃ and L₁, or R₄ and L₁, taken together, are C₁-C₅ alkylene or substituted alkylene;
G is selected from O, N, S; and
Z is a reactive group selected from the group consisting of thiols, disulfides, esters, thioesters, amines, anhydrides, hydrazines, aldehydes, ketones, boronic acids, carboxylic acids, azides, alkyl halides, alkenes, alkynes, alcohols, isocyanates, isothiocyanates, sulfonyl chlorides, epoxides, carbonates, hydroxymethyl phosphines, 2-iminothiolanes, and aziridines; or
(b) a construct having the structure:
A-L₂-B—Y—C-L₃-Z
wherein:
A is a biologically compatible material;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core 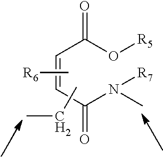
wherein:
R₅ is optionally present, and, when present, is selected from H, an alkali metal ion, an ammonium ion;
R₆ and R₇ are independently hydrogen, optionally substituted lower alkyl, optionally substituted aryl, or optionally substituted heteroaryl; and
Z is a reactive group selected from the group consisting of thiols, —SSR (R=aryl, heteroaryl), amines, azides, alkynes. Alkenes; or
(c) a construct having the structure:
A-L₂-B—Y—C-L₃-Z
wherein:
A is a biologically compatible material;
B and C are independently a covalent bond or a methylene unit optionally mono-substituted or di-substituted with lower alkyl, optionally substituted aryl, or optionally substituted heteroaryl;
L₂ and L₃ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from: 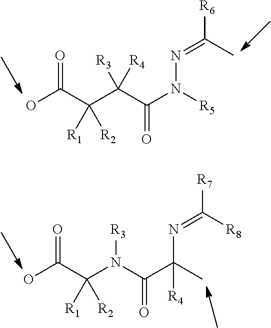
wherein:
R₁, R₂, R₃ and R₄ are independently hydrogen, optionally substituted lower alkyl, optionally substituted aryl, or optionally substituted heteroaryl; or, R₁ and R₃, taken together, are C₁-C₆ alkylene or substituted alkylene;
R₅ is optionally substituted lower alkyl;
R₆ is optionally substituted lower alkyl, optionally substituted aryl, or optionally substituted heteroaryl;
R₇ and R₈ are independently optionally substituted aryl, or optionally substituted heteroaryl, provided, however, that one of R₇ and R₈ is ortho-alkoxy substituted aryl; and
Z is selected from the group consisting of thiols, disulfides, esters, thioesters, amines, anhydrides, hydrazines, aldehydes, ketones, boronic acids, carboxylic acids, azides, alkyl halides, alkenes, alkynes, alcohols, isocyanates, isothiocyanates, sulfonyl chlorides, epoxides, carbonates, hydroxymethyl phosphines, 2-iminothiolanes, and aziridines.

11. The construct of claim 10 wherein said biologically compatible material A is selected from the group consisting of a biologically active molecule, a peptide, an oligopeptide, a protein, an antibody, a protein complex, a lipid, an oligosaccharide, a nucleic acid, a polyethylene glycol, a macrocycle, and an oligonucleotide.

12. The construct of claim 10 wherein said hydrolytically labile core Y is cleavable under physiological conditions.

13. The construct of claim 10 wherein said hydrolytically labile core Y is cleavable under conditions existing in certain intracellular compartments, in malignant cells, in foreign cells (e.g., parasites), or in cells undergoing specific changes related to disease states (e.g., inflammation, apoptosis, starvation, and the like).

14. The construct of claim 10 wherein L₂ and L₃ are each independently selected from the group consisting of a covalent bond, alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof.

15. A composition comprising the construct of claim 10 and a pharmaceutically acceptable carrier therefore.

16. The composition of claim 15 further comprising the construct D-X″, wherein:
D of the construct D-X″ is a biologically compatible material, and
X″ is a reactive group which is reactive with said reactive group Z.

17. A construct having the structure:
(a) A-L₂-B—Y—C-L₄-D
wherein:
A and D are independently biologically compatible materials containing at least 4 carbon atoms;
B and C are independently a covalent bond or a methylene unit optionally mono-substituted or di-substituted with lower alkyl, optionally substituted aryl, or optionally substituted heteroaryl;
L₂ and L₄ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R′)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from: 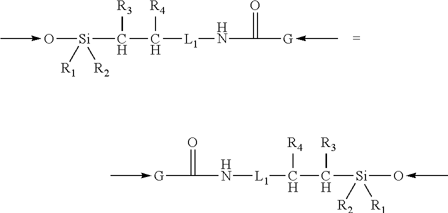
wherein:
R₁ and R₂ are independently optionally substituted lower alkyl, optionally substituted aryl, or optionally substituted heteroaryl;
R₃ and R₄ are independently hydrogen or optionally substituted lower alkyl, or, R₃ and R₄, taken together, are C₁-C₅ alkylene or substituted alkylene;
L₁ is a covalent bond, or a linker selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, and combinations of any two or more thereof, or R₃ and L₁, or R₄ and L₁, taken together, are C₁-C₅ alkylene or substituted alkylene; and
G is selected from O, N, S; or
(b) a construct having the structure:
A-L₂-B—Y—C-L₄-D
wherein:
A and D are independently biologically compatible materials;
B and C are independently a covalent bond or a methylene unit optionally mono-substituted or di-substituted with lower alkyl, optionally substituted aryl, or optionally substituted heteroaryl;
L₂ and L₄ are each independently a covalent bond or a bi-functional moiety selected from the group consisting of alkylene, substituted alkylene, heteroalkylene, substituted heteroalkylene, alkenylene, substituted alkenylene, heteroalkenylene, substituted heteroalkenylene, alkynylene, substituted alkynylene, heteroalkynylene, substituted heteroalkynylene, arylene, substituted arylene, heteroarylene, substituted heteroarylene, cyloalkylene, substituted cycloalkylene, heterocyloalkylene, substituted heterocycloalkylene, a linear core system, —O—, —O—(CR′₂)_{z}—, —S—, —NR′—, —NH—(CR′₂)_{z}—, —N═N—, —C(O)—, —C(O)NR′—, —O—C(O)—, —O—C(O)—O—, —O—C(O)—NR′—, —NR′—C(O)—, —NR′—C(O)—O—, —NR′—C(O)—NR′—, —S—C(O)—, —S—C(O)—O—, —S—C(O)—NR′—, —S(O)—, —S(O)₂—, —O—S(O)₂—, —O—S(O)₂—O—, —O—S(O)₂—NR′—, —O—S(O)—, —O—S(O)—O—, —O—S(O)—NR′—, —O—NR′—C(O)—, —O—NR′—C(O)—O—, —O—NR′—C(O)—NR′—, —NR′—O—C(O)—, —NR′—O—C(O)—O—, —NR′—O—C(O)—NR′—, —O—NR′—C(S)—, —O—NR′—C(S)—O—, —O—NR′—C(S)—NR′—, —NR′—O—C(S)—, —NR′—O—C(S)—O—, —NR′—O—C(S)—NR′—, —O—C(S)—, —O—C(S)—O—, —O—C(S)—NR′—, —NR′—C(S)—, —NR′—C(S)—O—, —NR′—C(S)—NR′—, —S—S(O)₂—, —S—S(O)₂—O—, —S—S(O)₂—NR′—, —NR′—O—S(O)—, —NR′—O—S(O)—O—, —NR′—O—S(O)—NR′—, —NR′—O—S(O)₂—, —NR′—O—S(O)₂—O—, —NR′—O—S(O)₂—NR′—, —O—NR′—S(O)—, —O—NR′—S(O)—O—, —O—NR′—S(O)—NR′—, —O—NR′—S(O)₂—O—, —O—NR′—S(O)₂—NR′—, —O—NR′—S(O)₂—, —O—P(O)(R)₂—, —S—P(O)(R′)₂—, and —NR′—P(O)(R′)₂—, wherein each R′ is independently hydrogen, substituted or unsubstituted alkyl, substituted or unsubstituted alkenyl, substituted or unsubstituted alkynyl, or substituted or unsubstituted cycloalkyl, and z is 1-10, and combinations of any two or more thereof; and
Y is a hydrolytically labile core selected from: 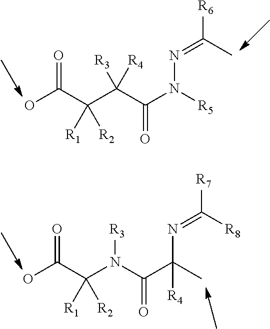
wherein:
R₁, R₂, R₃ and R₄ are independently hydrogen, optionally substituted lower alkyl, optionally substituted aryl, or optionally substituted heteroaryl; or, R₁ and R₃, taken together, are C₁-C₆ alkylene or substituted alkylene;
R₅ is optionally substituted lower alkyl;
R₆ is optionally substituted lower alkyl, optionally substituted aryl, or optionally substituted heteroaryl; and
R₇ and R₈ are independently optionally substituted aryl, or optionally substituted heteroaryl, provided, however, that one of R₇ and R₈ is ortho-alkoxy substituted aryl.

18. The construct of claim 17 wherein said biologically compatible materials A and D are independently selected from the group consisting of a biologically active molecule, a peptide, an oligopeptide, a protein, an antibody, a protein complex, an oligosaccharide, a nucleic acid, a polyethylene glycol, an amphiphilic macrocycle, a lipid, and an oligonucleotide.

19. The construct of claim 17 wherein said biologically compatible materials A and D are independently selected from the group consisting of a cationic peptide, a cyclodextrin, an oligonucleotide, polyethylene glycol, a polyethylene glycol derivative, a sterol, a sterol derivative, cholic acid, a cholic acid derivative, a secosteroid, a monoglyceride, a diglyceride, a phospholipid, a phospholipid derivative, a fatty acid, a fatty acid derivative, sirolimus, everolimus, paclitaxel, docetaxel, camptothecin, epothilone, an epothilone derivative, doxorubicin, maytansinoid, ansamycin, a retinoid, a tocopherol, a tocotrienol, biotin, a vitamin B compound, duocarmycin, a duocarmycin derivative, auristatin, and an auristatin derivative.

20. The construct of claim 17 wherein said hydrolytically labile core Y is cleavable under physiological conditions.

21. The construct of claim 17 wherein said hydrolytically labile core Y is cleavable under conditions existing in certain intracellular compartments, in malignant cells, in foreign cells (e.g., parasites), or in cells undergoing specific changes related to disease states (e.g., inflammation, apoptosis, starvation, and the like).

22. The construct of claim 17 wherein L₄ is produced by the reaction of E of the construct D-E with Z of the construct A-L₂-B—Y—C-L₃-Z, wherein E is a reactive group which reacts with Z.

23. The construct of claim 22 wherein said reactive groups E and Z are independently selected from the group consisting of thiols, disulfides, esters, thioesters, amines, anhydrides, hydrazines, aldehydes, ketones, boronic acids, azides, alkyl halides, alkenes, alkynes, alcohols, isocyanates, isothiocyanates, sulfonyl chlorides, epoxides, carbonates, hydroxymethyl phosphines, 2-iminothiolanes, and aziridines.

24. The construct of claim 22 wherein the linkage between L₄ and D results from the reaction of E with Z under physiological conditions.

25. The construct of claim 17 wherein the linkage between L₄ and D is a disulfide, an amide, a triazole, a urea, a thiourea, a thioether, a sulfonamide, a boronate, an amine, an amidine, a carbamate, a guanidine, an imine, or a hydrazone.

26. The construct of claim 17 wherein the linkage between L₄ and D is a disulfide, a silyl ether, a hydrazone, a ketal, an acetal, a maleate amide, a boronate, or an imine.

27. The construct of claim 26 wherein the linkage between L₄ and D is cleavable under physiological conditions.

28. A composition comprising the construct of claim 17 and a pharmaceutically acceptable carrier therefore.

29. A method of delivering a biologically compatible material A, or derivative thereof, to a subject in need thereof, said method comprising administering to said subject an effective amount of a composition comprising a construct according to claim 17, wherein Y is further characterized by cleaving under physiological conditions to produce constructs containing A-L₂ and/or D-L₄ as fragments thereof.

30. A method of delivering a biologically compatible material A, or derivative thereof, to a subject in need thereof, said method comprising administering to said subject an effective amount of a combination comprising a construct according to claim 10 and the construct D-E, wherein:
E is a reactive group characterized by reacting with Z to form a linkage between L₄ and D, and
D is a biologically compatible material.

31. A method of delivering a biologically compatible material to a subject in need thereof, said method comprising administering to said subject an effective amount of a construct according to claim 17.

32. A method of delivering a biologically compatible material D, or derivative thereof, to a subject in need thereof, said method comprising administering to said subject an effective amount of a combination comprising a construct according to claim 10 and the construct D-E, wherein:
E is a reactive group characterized by:
reacting with Z under physiological conditions to form a linkage between L₄ and D, or
forming a covalent bond between L₄ and D which bond is cleavable under physiological conditions, and
D is a biologically compatible material.

33. A method of modifying a biologically compatible material A with a modifying agent D, or modifying a biologically compatible material D with a modifying agent A,
said method comprising contacting the construct according to claim 10 with the construct D-E under conditions suitable for the formation of the construct A-L₂-B—Y—C-L₄-D.

34. A method of preparing a construct according to claim 17, said method comprising contacting A-L₂-B—Y—C-L₃-Z with D-E under conditions suitable for the formation of the construct A-L₂-B—Y—C-L₄-D.

35. A method for releasing active component A from the construct according to claim 17, or releasing active component D from said construct,
said method comprising subjecting said construct to physiological conditions suitable to cleave the hydrolytically labile core, Y, or the bond adjacent to at least one of the L₂, L₃ or the L₄ linkages.
